(19) United States
(12) Patent Application Publication
(10) Pub. No.: US 2024/0215908 A1
(43) Pub. Date: Jul. 4, 2024

(54) SYSTEMS AND METHODS FOR DETERMINING A SLEEP STAGE OF AN INDIVIDUAL

(71) Applicant: RESMED SENSOR TECHNOLOGIES LIMITED, Dublin (IE)

(72) Inventors: Anna RICE, Dublin (IE); Graeme Alexander LYON, Dublin (IE); Niall Andrew FOX, Dublin (IE)

(21) Appl. No.: 18/563,777

(22) PCT Filed: May 22, 2022
(86) PCT No.: PCT/IB2022/054772

§ 371 Date: Nov. 22, 2023

Related U.S. Application Data
(60) Provisional application No. 63/192,343, filed on May 24, 2021.

(51) Int. Cl. A61B 5/00; A61M 16/00; A61M 16/06; A61M 16/10

(52) U.S. Cl. CPC A61B 5/4812; A61B 5/4809; A61B 5/7246; A61B 5/7264; A61B 5/7282; A61M 16/0066; A61M 16/0616; A61M 16/10

(57) ABSTRACT

A method of determining a sleep stage of an individual comprises identifying features associated with a current epoch of the sleep session, determining a plurality of sleep stage probabilities based on the features, identifying events experienced by the individual during the current epoch, and adjusting each of the plurality of sleep stage probabilities based on events experienced by the individual during the current epoch or a prior epoch, and/or a sleep stage previously determined for the prior epoch. The features include at least one feature associated with a flow of pressurized air from a respiratory therapy system used by the individual, at least one feature associated with a respiration rate of the individual, and/or at least one feature associated with a time of the sleep session. Each sleep stage probability corresponds to a respective one of a plurality of potential sleep stages during the current epoch of the sleep session.

### CROSS-REFERENCE TO RELATED APPLICATIONS

This application is a U.S. National Stage of International Application No. PCT/IB2022/054772, filed on May 22, 2022, which claims the benefit of, and priority to, U.S. Provisional Patent Application No. 63/192,343 filed on May 24, 2021, each of which is hereby incorporated by reference herein in its entirety.

### TECHNICAL FIELD

The present disclosure relates generally to systems and methods for determining the sleep stage of an individual during a sleep session, and more particularly, to systems and methods for determining the sleep stage of an individual during a sleep session based on flow data, respiratory data, and respiratory events.

### BACKGROUND

Many individuals suffer from sleep-related and/or respiratory-related disorders such as, for example, Sleep Disordered Breathing (SDB), which can include Obstructive Sleep Apnea (OSA), Central Sleep Apnea (CSA), other types of apneas such as mixed apneas and hypopneas, Respiratory Effort Related Arousal (RERA), and snoring. In some cases, these disorders manifest, or manifest more pronouncedly, when the individual is in a particular lying/sleeping position. These individuals may also suffer from other health conditions (which may be referred to as comorbidities), such as insomnia (e.g., difficulty initiating sleep, frequent or prolonged awakenings after initially falling asleep, and/or an early awakening with an inability to return to sleep), Periodic Limb Movement Disorder (PLMD), Restless Leg Syndrome (RLS), Cheyne-Stokes Respiration (CSR), respiratory insufficiency, Obesity Hyperventilation Syndrome (OHS), Chronic Obstructive Pulmonary Disease (COPD), Neuromuscular Disease (NMD), rapid eye movement (REM) behavior disorder (also referred to as RBD), dream enactment behavior (DEB), hypertension, diabetes, stroke, and chest wall disorders.

These disorders are often treated using a respiratory therapy system (e.g., a continuous positive airway pressure (CPAP) system), which delivers pressurized air to aid in preventing the individual's airway from narrowing or collapsing during sleep. While respiratory therapy systems can be configured to detect sleep-disordered breathing (SDB) events, such as apneas and hypopneas, in real time, they may often incorrectly report SDB events (e.g., the calculated AHI) based on the user not being asleep or the user being in an unexpected stage of sleep. Thus, new systems and methods are needed for determining the sleep stage of the individual, in particular during a sleep session in which the individual is using a respiratory therapy system. The present disclosure is directed to solving these and other problems.

### SUMMARY

According to some implementations of the present disclosure, a method of determining a sleep stage of an individual comprises receiving data associated with a sleep session of the individual. The sleep session is divided into a plurality of epochs. The method also includes analyzing the received data to identify one or more features associated with a current epoch of the sleep session. The one or more features includes (i) at least one feature associated with a flow of pressurized air from a respiratory therapy system used by the individual during the sleep session, (ii) at least one feature associated with a respiration rate of the individual, (iii) at least one feature associated with a temporal location of the current epoch within the sleep session, or (iv) any combination of (i)-(iii). The method also includes determining, based on at least the one or more features, a plurality of sleep stage probabilities. Each sleep stage probability corresponds to a respective one of a plurality of potential sleep stages during the current epoch of the sleep session. The method also includes analyzing the data to identify events experienced by the individual during the current epoch of the sleep session. The method also includes adjusting each of the plurality of sleep stage probabilities based on (i) events experienced by the individual during the current epoch of the sleep session, (ii) events experienced by the individual during a prior epoch of the sleep session, (iii) events experienced by the individual during a subsequent epoch of the sleep session, (iv) a sleep stage determined for the prior epoch of the sleep session, (v) a sleep sage determined for the subsequent epoch of the sleep session, or (vi) any combination of (i)-(v).

According to some implementations of the present disclosure, a system for determining a sleep stage of an individual comprises an electronic interface, a control system, and a memory. The electronic interface is configured to receive data associated with a sleep session of the individual. The memory stores machine-readable instructions. The control system includes one or more processors configured to execute the machine-readable instructions to execute a method. The method includes receiving data associated with a sleep session of the individual. The sleep session is divided into a plurality of epochs. The method also includes analyzing the received data to identify one or more features associated with a current epoch of the sleep session. The one or more features includes (i) at least one feature associated with a flow of pressurized air from a respiratory therapy system used by the individual during the sleep session, (ii) at least one feature associated with a respiration rate of the individual, (iii) at least one feature associated with a time of the sleep session, or (iv) any combination of (i)-(iii). The method also includes determining, based on at least the one or more features, a plurality of sleep stage probabilities. Each sleep stage probability corresponds to a respective one of a plurality of potential sleep stages during the current epoch of the sleep session. The method also includes analyzing the data to identify events experienced by the individual during the current epoch of the sleep session. The method also includes adjusting each of the plurality of sleep stage probabilities based on (i) events experienced by the individual during the current epoch of the sleep session, (ii) events experienced by the individual during a prior epoch of the sleep session, or (iii) a sleep stage previously determined for the prior epoch of the sleep session.

According to some implementations of the present disclosure, a method of determining a sleep stage of an individual comprises receiving data associated with a sleep session of the individual. The sleep session is divided into a plurality of epochs. The method also includes analyzing the received data to identify one or more features associated with a current epoch of the sleep session. The one or more features includes (i) at least one feature associated with a flow of pressurized air from a respiratory therapy system used by the individual during the sleep session, (ii) at least one feature associated with a respiration rate of the individual, (iii) at least one feature associated with a temporal location of the current epoch within the sleep session, or (iv) any combination of (i)-(iii). The method also includes determining, based on at least the one or more features, a plurality of sleep stage probabilities. Each sleep stage probability corresponds to a respective one of a plurality of potential sleep stages during the current epoch of the sleep session.

According to some implementations of the present disclosure, a system for determining a sleep stage of an individual comprises an electronic interface, a control system, and a memory. The electronic interface is configured to receive data associated with a sleep session of the individual. The memory stores machine-readable instructions. The control system includes one or more processors configured to execute the machine-readable instructions to execute a method. The method includes receiving data associated with a sleep session of the individual. The sleep session is divided into a plurality of epochs. The method also includes analyzing the received data to identify one or more features associated with a current epoch of the sleep session. The one or more features includes (i) at least one feature associated with a flow of pressurized air from a respiratory therapy system used by the individual during the sleep session, (ii) at least one feature associated with a respiration rate of the individual, (iii) at least one feature associated with a time of the sleep session, or (iv) any combination of (i)-(iii). The method also includes determining, based on at least the one or more features, a plurality of sleep stage probabilities. Each sleep stage probability corresponds to a respective one of a plurality of potential sleep stages during the current epoch of the sleep session.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

FIG. 1 is a functional block diagram of a respiratory therapy system, according to some implementations of the present disclosure;

FIG. 2 is a perspective view of the respiratory therapy system of FIG. 1, a user of the respiratory therapy system, and a bed partner of the user, according to some implementations of the present disclosure;

FIG. 3 illustrates an exemplary timeline for a sleep session, according to some implementations of the present disclosure;

FIG. 4 illustrates an exemplary hypnogram associated with the sleep session of FIG. 3, according to some implementations of the present disclosure;

FIG. 5 is a functional block diagram of an algorithm for determining sleep stages of an individual during a sleep session; and

FIG. 6 is a process flow diagram for a method of determining a sleep stage of an individual, according to some implementations of the present disclosure.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

The present disclosure is described with reference to the attached figures, where like reference numerals are used throughout the figures to designate similar or equivalent elements. The figures are not drawn to scale, and are provided merely to illustrate the instant disclosure. Several aspects of the disclosure are described below with reference to example applications for illustration.

Many individuals suffer from sleep-related and/or respiratory disorders, such as Sleep Disordered Breathing (SDB) such as Obstructive Sleep Apnea (OSA), Central Sleep Apnea (CSA) and other types of apneas, Respiratory Effort Related Arousal (RERA), snoring, Cheyne-Stokes Respiration (CSR), respiratory insufficiency, Obesity Hyperventilation Syndrome (OHS), Chronic Obstructive Pulmonary Disease (COPD), Periodic Limb Movement Disorder (PLMD), Restless Leg Syndrome (RLS), Neuromuscular Disease (NMD), and chest wall disorders.

Obstructive Sleep Apnea (OSA), a form of Sleep Disordered Breathing (SDB), is characterized by events including occlusion or obstruction of the upper air passage during sleep resulting from a combination of an abnormally small upper airway and the normal loss of muscle tone in the region of the tongue, soft palate and posterior oropharyngeal wall. More generally, an apnea generally refers to the cessation of breathing caused by blockage of the air (Obstructive Sleep Apnea) or the stopping of the breathing function (often referred to as Central Sleep Apnea). CSA results when the brain temporarily stops sending signals to the muscles that control breathing. Typically, the individual will stop breathing for between about 15 seconds and about 30 seconds during an obstructive sleep apnea event.

Other types of apneas include hypopnea, hyperpnea, and hypercapnia. Hypopnea is generally characterized by slow or shallow breathing caused by a narrowed airway, as opposed to a blocked airway. Hyperpnea is generally characterized by an increase depth and/or rate of breathing. Hypercapnia is generally characterized by elevated or excessive carbon dioxide in the bloodstream, typically caused by inadequate respiration.

A Respiratory Effort Related Arousal (RERA) event is typically characterized by an increased respiratory effort for ten seconds or longer leading to arousal from sleep and which does not fulfill the criteria for an apnea or hypopnea event. RERAs are defined as a sequence of breaths characterized by increasing respiratory effort leading to an arousal from sleep, but which does not meet criteria for an apnea or hypopnea. These events fulfil the following criteria: (1) a pattern of progressively more negative esophageal pressure, terminated by a sudden change in pressure to a less negative level and an arousal, and (2) the event lasts ten seconds or longer. In some implementations, a Nasal Cannula/Pressure Transducer System is adequate and reliable in the detection of RERAs. A RERA detector may be based on a real flow signal derived from a respiratory therapy device. For example, a flow limitation measure may be determined based on a flow signal. A measure of arousal may then be derived as a function of the flow limitation measure and a measure of sudden increase in ventilation. One such method is described in WO 2008/138040 and U.S. Pat. No. 9,358,353, assigned to ResMed Ltd., the disclosure of each of which is hereby incorporated by reference herein in their entireties.

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterized by repetitive de-oxygenation and re-oxygenation of the arterial blood.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common, such as increased resistance to air movement, extended expiratory phase of respiration, and loss of the normal elasticity of the lung. COPD encompasses a group of lower airway diseases that have certain characteristics in common, such as increased resistance to air movement, extended expiratory phase of respiration, and loss of the normal elasticity of the lung.

Neuromuscular Disease (NMD) encompasses many diseases and ailments that impair the functioning of the muscles either directly via intrinsic muscle pathology, or indirectly via nerve pathology. Chest wall disorders are a group of thoracic deformities that result in inefficient coupling between the respiratory muscles and the thoracic cage.

These and other disorders are characterized by particular events (e.g., snoring, an apnea, a hypopnea, a restless leg, a sleeping disorder, choking, an increased heart rate, labored breathing, an asthma attack, an epileptic episode, a seizure, or any combination thereof) that occur when the individual is sleeping.

The Apnea-Hypopnea Index (AHI) is an index used to indicate the severity of sleep apnea during a sleep session. The AHI is calculated by dividing the number of apnea and/or hypopnea events experienced by the user during the sleep session by the total number of hours of sleep in the sleep session. The event can be, for example, a pause in breathing that lasts for at least 10 seconds. An AHI that is less than 5 is considered normal. An AHI that is greater than or equal to 5, but less than 15 is considered indicative of mild sleep apnea. An AHI that is greater than or equal to 15, but less than 30 is considered indicative of moderate sleep apnea. An AHI that is greater than or equal to 30 is considered indicative of severe sleep apnea. In children, an AHI that is greater than 1 is considered abnormal. Sleep apnea can be considered “controlled” when the AHI is normal, or when the AHI is normal or mild. The AHI can also be used in combination with oxygen desaturation levels to indicate the severity of Obstructive Sleep Apnea.

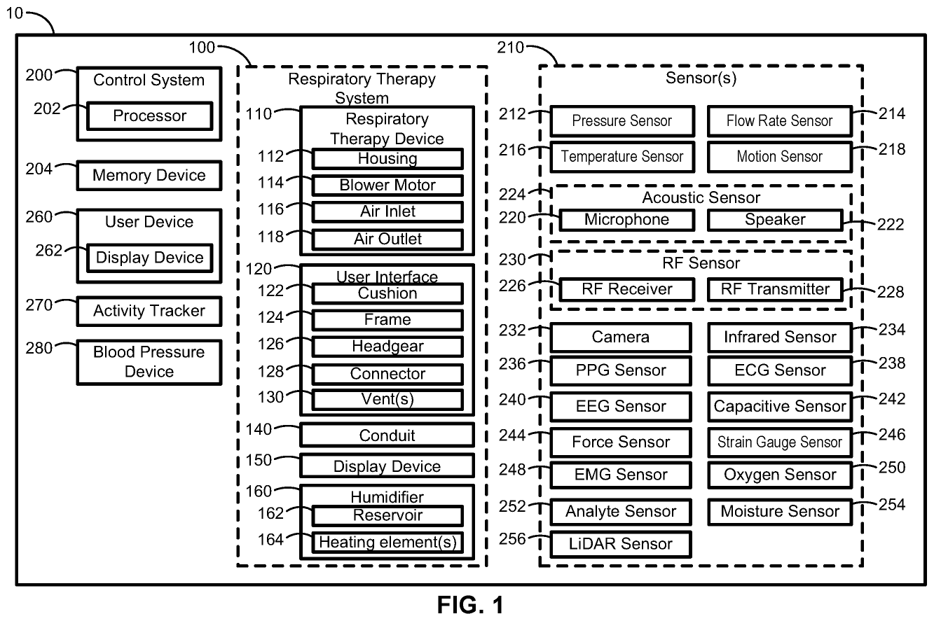

Referring to FIG. 1, a system 10, according to some implementations of the present disclosure, is illustrated. The system 10 includes a respiratory therapy system 100, a control system 200, a memory device 204, and one or more sensors 210. The system 10 may additionally or alternatively include a user device 260, an activity tracker 270, and a blood pressure device 280. The system 10 can be used to determine sleep stages of a user during a sleep session.

The respiratory therapy system 100 includes a respiratory pressure therapy (RPT) device 110 (referred to herein as respiratory therapy device 110), a user interface 120 (also referred to as a mask or a patient interface), a conduit 140 (also referred to as a tube or an air circuit), a display device 150, and a humidifier 160. Respiratory pressure therapy refers to the application of a supply of air to an entrance to a user's airways at a controlled target pressure that is nominally positive with respect to atmosphere throughout the user's breathing cycle (e.g., in contrast to negative pressure therapies such as the tank ventilator or cuirass). The respiratory therapy system 100 is generally used to treat individuals suffering from one or more sleep-related respiratory disorders (e.g., obstructive sleep apnea, central sleep apnea, or mixed sleep apnea).

The respiratory therapy system 100 can be used, for example, as a ventilator or as a positive airway pressure (PAP) system, such as a continuous positive airway pressure (CPAP) system, an automatic positive airway pressure system (APAP), a bi-level or variable positive airway pressure system (BPAP or VPAP), or any combination thereof. The CPAP system delivers a predetermined air pressure (e.g., determined by a sleep physician) to the user. The APAP system automatically varies the air pressure delivered to the user based on, for example, respiration data associated with the user. The BPAP or VPAP system is configured to deliver a first predetermined pressure (e.g., an inspiratory positive airway pressure or IPAP) and a second predetermined pressure (e.g., an expiratory positive airway pressure or EPAP) that is lower than the first predetermined pressure.

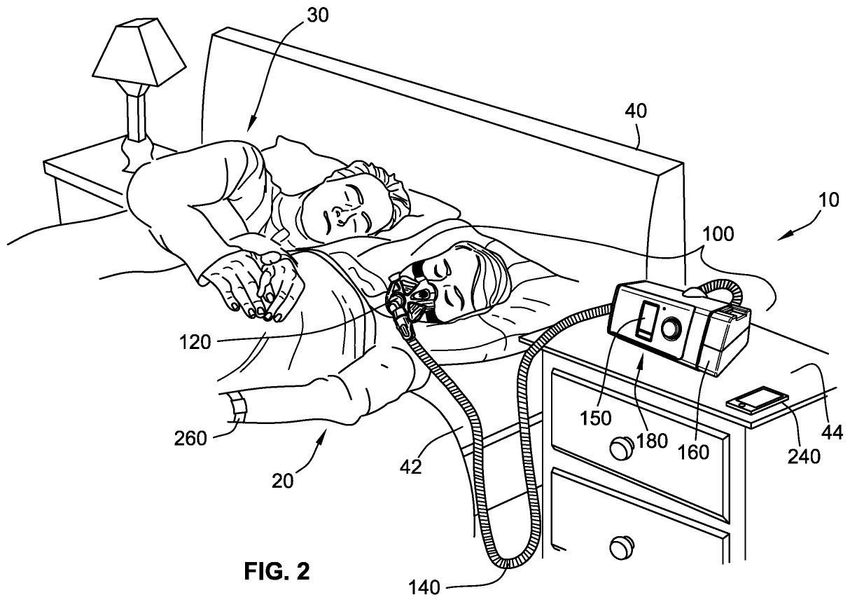

As shown in FIG. 2, the respiratory therapy system 100 can be used to treat a user 20. In this example, the user 20 of the respiratory therapy system 100 and a bed partner 30 are located in a bed 40 and are laying on a mattress 42. The user interface 120 can be worn by the user 20 during a sleep session. The respiratory therapy system 100 generally aids in increasing the air pressure in the throat of the user 20 to aid in preventing the airway from closing and/or narrowing during sleep. The respiratory therapy device 110 can be positioned on a nightstand 44 that is directly adjacent to the bed 40 as shown in FIG. 2, or more generally, on any surface or structure that is generally adjacent to the bed 40 and/or the user 20.

Referring back to FIG. 1, the respiratory therapy device 110 is generally used to generate pressurized air that is delivered to a user (e.g., using one or more motors that drive one or more compressors). In some implementations, the respiratory therapy device 110 generates continuous constant air pressure that is delivered to the user. In other implementations, the respiratory therapy device 110 generates two or more predetermined pressures (e.g., a first predetermined air pressure and a second predetermined air pressure). In still other implementations, the respiratory therapy device 110 generates a variety of different air pressures within a predetermined range. For example, the respiratory therapy device 110 can deliver at least about 6 cmH₂O, at least about 10 cmH₂O, at least about 20 cmH₂O, between about 6 cmH₂O and about 10 cmH₂O, between about 7 cmH₂O and about 12 cmH₂O, etc. The respiratory therapy device 110 can also deliver pressurized air at a predetermined flow rate between, for example, about −20 L/min and about 150 L/min, while maintaining a positive pressure (relative to the ambient pressure).

The respiratory therapy device 110 includes a housing 112, a blower motor 114, an air inlet 116, and an air outlet 118. The blower motor 114 is at least partially disposed or integrated within the housing 112. The blower motor 114 draws air from outside the housing 112 (e.g., atmosphere) via the air inlet 116 and causes pressurized air to flow through the humidifier 160, and through the air outlet 118. In some implementations, the air inlet 116 and/or the air outlet 118 include a cover that is moveable between a closed position and an open position (e.g., to prevent or inhibit air from flowing through the air inlet 116 or the air outlet 118). The housing 112 can also include a vent to allow air to pass through the housing 112 to the air inlet 116. As described below, the conduit 140 is coupled to the air outlet 118 of the respiratory therapy device 110.

The user interface 120 engages a portion of the user's face and delivers pressurized air from the respiratory therapy device 110 to the user's airway to aid in preventing the airway from narrowing and/or collapsing during sleep. This may also increase the user's oxygen intake during sleep. Generally, the user interface 120 engages the user's face such that the pressurized air is delivered to the user's airway via the user's mouth, the user's nose, or both the user's mouth and nose. Together, the respiratory therapy device 110, the user interface 120, and the conduit 140 form an air pathway fluidly coupled with an airway of the user. The pressurized air also increases the user's oxygen intake during sleep. Depending upon the therapy to be applied, the user interface 120 may form a seal, for example, with a region or portion of the user's face, to facilitate the delivery of gas at a pressure at sufficient variance with ambient pressure to effect therapy, for example, at a positive pressure of about 10 cm H₂O relative to ambient pressure. For other forms of therapy, such as the delivery of oxygen, the user interface may not include a seal sufficient to facilitate delivery to the airways of a supply of gas at a positive pressure of about 10 cmH₂O.

The user interface 120 can include, for example, a cushion 122, a frame 124, a head gear 126, connector 128, and one or more vents 130. The cushion 122 and the frame 124 define a volume of space around the mouth and/or nose of the user. When the respiratory therapy system 100 is in use, this volume space receives pressurized air (e.g., from the respiratory therapy device 110 via the conduit 140) for passage into the airway(s) of the user. The headgear 126 is generally used to aid in positioning and/or stabilizing the user interface 120 on a portion of the user (e.g., the face), and along with the cushion 122 (which, for example, can comprise silicone, plastic, foam, etc.) aids in providing a substantially air-tight seal between the user interface 120 and the user 20. In some implementations the headgear 126 includes one or more straps (e.g., including hook and loop fasteners). The connector 128 is generally used to couple (e.g., connect and fluidly couple) the conduit 140 to the cushion 122 and/or frame 124. Alternatively, the conduit 140 can be directly coupled to the cushion 122 and/or frame 124 without the connector 128. The vent 130 can be used for permitting the escape of carbon dioxide and other gases exhaled by the user 20. The user interface 120 generally can include any suitable number of vents (e.g., one, two, five, ten, etc.).

As shown in FIG. 2, in some implementations, the user interface 120 is a facial mask (e.g., a full face mask) that covers at least a portion of the nose and mouth of the user 20. Alternatively, the user interface 120 can be a nasal mask that provides air to the nose of the user or a nasal pillow mask that delivers air directly to the nostrils of the user 20. In other implementations, the user interface 120 includes a mouthpiece (e.g., a night guard mouthpiece molded to conform to the teeth of the user, a mandibular repositioning device, etc.).

Referring back to FIG. 1, the conduit 140 (also referred to as an air circuit or tube) allows the flow of air between components of the respiratory therapy system 100, such as between the respiratory therapy device 110 and the user interface 120. In some implementations, there can be separate limbs of the conduit for inhalation and exhalation. In other implementations, a single limb conduit is used for both inhalation and exhalation.

The conduit 140 includes a first end that is coupled to the air outlet 118 of the respiratory therapy device 110. The first end can be coupled to the air outlet 118 of the respiratory therapy device 110 using a variety of techniques (e.g., a press fit connection, a snap fit connection, a threaded connection, etc.). In some implementations, the conduit 140 includes one or more heating elements that heat the pressurized air flowing through the conduit 140 (e.g., heat the air to a predetermined temperature or within a range of predetermined temperatures). Such heating elements can be coupled to and/or imbedded in the conduit 140. In such implementations, the first end can include an electrical contact that is electrically coupled to the respiratory therapy device 110 to power the one or more heating elements of the conduit 140. For example, the electrical contact can be electrically coupled to an electrical contact of the air outlet 118 of the respiratory therapy device 110. In this example, electrical contact of the conduit 140 can be a male connector and the electrical contact of the air outlet 118 can be female connector, or, alternatively, the opposite configuration can be used.

The display device 150 is generally used to display image(s) including still images, video images, or both and/or information regarding the respiratory therapy device 110. For example, the display device 150 can provide information regarding the status of the respiratory therapy device 110 (e.g., whether the respiratory therapy device 110 is on/off, the pressure of the air being delivered by the respiratory therapy device 110, the temperature of the air being delivered by the respiratory therapy device 110, etc.) and/or other information (e.g., a sleep score and/or a therapy score, also referred to as a myAir™ score, such as described in WO 2016/061629 and U.S. Patent Pub. No. 2017/0311879, which are hereby incorporated by reference herein in their entireties, the current date/time, personal information for the user 20, etc.). In some implementations, the display device 150 acts as a human-machine interface (HMI) that includes a graphic user interface (GUI) configured to display the image(s) as an input interface. The display device 150 can be an LED display, an OLED display, an LCD display, or the like. The input interface can be, for example, a touchscreen or touch-sensitive substrate, a mouse, a keyboard, or any sensor system configured to sense inputs made by a human user interacting with the respiratory therapy device 110.

The humidifier 160 is coupled to or integrated in the respiratory therapy device 110 and includes a reservoir 162 for storing water that can be used to humidify the pressurized air delivered from the respiratory therapy device 110. The humidifier 160 includes a one or more heating elements 164 to heat the water in the reservoir to generate water vapor. The humidifier 160 can be fluidly coupled to a water vapor inlet of the air pathway between the blower motor 114 and the air outlet 118, or can be formed in-line with the air pathway between the blower motor 114 and the air outlet 118. For example, air flows from the air inlet 116 through the blower motor 114, and then through the humidifier 160 before exiting the respiratory therapy device 110 via the air outlet 118.

While the respiratory therapy system 100 has been described herein as including each of the respiratory therapy device 110, the user interface 120, the conduit 140, the display device 150, and the humidifier 160, more or fewer components can be included in a respiratory therapy system according to implementations of the present disclosure. For example, a first alternative respiratory therapy system includes the respiratory therapy device 110, the user interface 120, and the conduit 140. As another example, a second alternative system includes the respiratory therapy device 110, the user interface 120, and the conduit 140, and the display device 150. Thus, various respiratory therapy systems can be formed using any portion or portions of the components shown and described herein and/or in combination with one or more other components.

The control system 200 includes one or more processors 202 (hereinafter, processor 202). The control system 200 is generally used to control (e.g., actuate) the various components of the system 10 and/or analyze data obtained and/or generated by the components of the system 10. The processor 202 can be a general or special purpose processor or microprocessor. While one processor 202 is illustrated in FIG. 1, the control system 200 can include any number of processors (e.g., one processor, two processors, five processors, ten processors, etc.) that can be in a single housing, or located remotely from each other. The control system 200 (or any other control system) or a portion of the control system 200 such as the processor 202 (or any other processor(s) or portion(s) of any other control system), can be used to carry out one or more steps of any of the methods described and/or claimed herein. The control system 200 can be coupled to and/or positioned within, for example, a housing of the user device 260, a portion (e.g., the respiratory therapy device 110) of the respiratory therapy system 100, and/or within a housing of one or more of the sensors 210. The control system 200 can be centralized (within one such housing) or decentralized (within two or more of such housings, which are physically distinct). In such implementations including two or more housings containing the control system 200, the housings can be located proximately and/or remotely from each other.

The memory device 204 stores machine-readable instructions that are executable by the processor 202 of the control system 200. The memory device 204 can be any suitable computer readable storage device or media, such as, for example, a random or serial access memory device, a hard drive, a solid state drive, a flash memory device, etc. While one memory device 204 is shown in FIG. 1, the system 10 can include any suitable number of memory devices 204 (e.g., one memory device, two memory devices, five memory devices, ten memory devices, etc.). The memory device 204 can be coupled to and/or positioned within a housing of a respiratory therapy device 110 of the respiratory therapy system 100, within a housing of the user device 260, within a housing of one or more of the sensors 210, or any combination thereof. Like the control system 200, the memory device 204 can be centralized (within one such housing) or decentralized (within two or more of such housings, which are physically distinct).

In some implementations, the memory device 204 stores a user profile associated with the user. The user profile can include, for example, demographic information associated with the user, biometric information associated with the user, medical information associated with the user, self-reported user feedback, sleep parameters associated with the user (e.g., sleep-related parameters recorded from one or more earlier sleep sessions), or any combination thereof. The demographic information can include, for example, information indicative of an age of the user, a gender of the user, a race of the user, a geographic location of the user, a relationship status, a family history of insomnia or sleep apnea, an employment status of the user, an educational status of the user, a socioeconomic status of the user, or any combination thereof. The medical information can include, for example, information indicative of one or more medical conditions associated with the user, medication usage by the user, or both. The medical information data can further include a multiple sleep latency test (MSLT) result or score and/or a Pittsburgh Sleep Quality Index (PSQI) score or value. The self-reported user feedback can include information indicative of a self-reported subjective sleep score (e.g., poor, average, excellent), a self-reported subjective stress level of the user, a self-reported subjective fatigue level of the user, a self-reported subjective health status of the user, a recent life event experienced by the user, or any combination thereof.

As described herein, the processor 202 and/or memory device 204 can receive data (e.g., physiological data and/or audio data) from the one or more sensors 210 such that the data for storage in the memory device 204 and/or for analysis by the processor 202. The processor 202 and/or memory device 204 can communicate with the one or more sensors 210 using a wired connection or a wireless connection (e.g., using an RF communication protocol, a Wi-Fi communication protocol, a Bluetooth communication protocol, over a cellular network, etc.). In some implementations, the system 10 can include an antenna, a receiver (e.g., an RF receiver), a transmitter (e.g., an RF transmitter), a transceiver, or any combination thereof. Such components can be coupled to or integrated a housing of the control system 200 (e.g., in the same housing as the processor 202 and/or memory device 204), or the user device 260.

The one or more sensors 210 include a pressure sensor 212, a flow rate sensor 214, temperature sensor 216, a motion sensor 218, a microphone 220, a speaker 222, a radio-frequency (RF) receiver 226, a RF transmitter 228, a camera 232, an infrared (IR) sensor 234, a photoplethysmogram (PPG) sensor 236, an electrocardiogram (ECG) sensor 238, an electroencephalography (EEG) sensor 240, a capacitive sensor 242, a force sensor 244, a strain gauge sensor 246, an electromyography (EMG) sensor 248, an oxygen sensor 250, an analyte sensor 252, a moisture sensor 254, a Light Detection and Ranging (LiDAR) sensor 256, or any combination thereof. Generally, each of the one or more sensors 210 are configured to output sensor data that is received and stored in the memory device 204 or one or more other memory devices.

While the one or more sensors 210 are shown and described as including each of the pressure sensor 212, the flow rate sensor 214, the temperature sensor 216, the motion sensor 218, the microphone 220, the speaker 222, the RF receiver 226, the RF transmitter 228, the camera 232, the IR sensor 234, the PPG sensor 236, the ECG sensor 238, the EEG sensor 240, the capacitive sensor 242, the force sensor 244, the strain gauge sensor 246, the EMG sensor 248, the oxygen sensor 250, the analyte sensor 252, the moisture sensor 254, and the LiDAR sensor 256, more generally, the one or more sensors 210 can include any combination and any number of each of the sensors described and/or shown herein.

As described herein, the system 10 generally can be used to generate physiological data associated with a user (e.g., a user of the respiratory therapy system 100) during a sleep session. The physiological data can be analyzed to generate one or more sleep-related parameters, which can include any parameter, measurement, etc. related to the user during the sleep session. The one or more sleep-related parameters that can be determined for the user 20 during the sleep session include, for example, an Apnea-Hypopnea Index (AHI) score, a sleep score, a flow signal, a respiration signal, a respiration rate, an inspiration amplitude, an expiration amplitude, an inspiration-expiration ratio, a number of events per hour, a pattern of events, a stage, pressure settings of the respiratory therapy device 110, a heart rate, a heart rate variability, movement of the user 20, temperature, EEG activity, EMG activity, arousal, snoring, choking, coughing, whistling, wheezing, or any combination thereof.

The one or more sensors 210 can be used to generate, for example, physiological data, audio data, or both. Physiological data generated by one or more of the sensors 210 can be used by the control system 200 to determine a sleep-wake signal associated with the user 20 during the sleep session and one or more sleep-related parameters. The sleep-wake signal can be indicative of one or more sleep states, including wakefulness, relaxed wakefulness, micro-awakenings, or distinct sleep stages such as, for example, a rapid eye movement (REM) stage, a first non-REM stage (often referred to as “N1”), a second non-REM stage (often referred to as “N2”), a third non-REM stage (often referred to as “N3”), or any combination thereof. Methods for determining sleep states and/or sleep stages from physiological data generated by one or more sensors, such as the one or more sensors 210, are described in, for example, WO 2014/047310, U.S. Patent Pub. No. 2014/0088373, WO 2017/132726, WO 2019/122413, WO 2019/122414, and U.S. Patent Pub. No. 2020/0383580 each of which is hereby incorporated by reference herein in its entirety.

In some implementations, the sleep-wake signal described herein can be timestamped to indicate a time that the user enters the bed, a time that the user exits the bed, a time that the user attempts to fall asleep, etc. The sleep-wake signal can be measured by the one or more sensors 210 during the sleep session at a predetermined sampling rate, such as, for example, one sample per second, one sample per 30 seconds, one sample per minute, etc. In some implementations, the sleep-wake signal can also be indicative of a respiration signal, a respiration rate, an inspiration amplitude, an expiration amplitude, an inspiration-expiration ratio, a number of events per hour, a pattern of events, pressure settings of the respiratory therapy device 110, or any combination thereof during the sleep session. The event(s) can include snoring, apneas, central apneas, obstructive apneas, mixed apneas, hypopneas, a mask leak (e.g., from the user interface 120), a restless leg, a sleeping disorder, choking, an increased heart rate, labored breathing, an asthma attack, an epileptic episode, a seizure, or any combination thereof. The one or more sleep-related parameters that can be determined for the user during the sleep session based on the sleep-wake signal include, for example, a total time in bed, a total sleep time, a sleep onset latency, a wake-after-sleep-onset parameter, a sleep efficiency, a fragmentation index, or any combination thereof. As described in further detail herein, the physiological data and/or the sleep-related parameters can be analyzed to determine one or more sleep-related scores.

Physiological data and/or audio data generated by the one or more sensors 210 can also be used to determine a respiration signal associated with a user during a sleep session. The respiration signal is generally indicative of respiration or breathing of the user during the sleep session. The respiration signal can be indicative of and/or analyzed to determine (e.g., using the control system 200) one or more sleep-related parameters, such as, for example, a respiration rate, a respiration rate variability, an inspiration amplitude, an expiration amplitude, an inspiration-expiration ratio, an occurrence of one or more events, a number of events per hour, a pattern of events, a sleep state, a sleet stage, an apnea-hypopnea index (AHI), pressure settings of the respiratory therapy device 110, or any combination thereof. The one or more events can include snoring, apneas, central apneas, obstructive apneas, mixed apneas, hypopneas, a mask leak (e.g., from the user interface 120), a cough, a restless leg, a sleeping disorder, choking, an increased heart rate, labored breathing, an asthma attack, an epileptic episode, a seizure, increased blood pressure, or any combination thereof. Many of the described sleep-related parameters are physiological parameters, although some of the sleep-related parameters can be considered to be non-physiological parameters. Other types of physiological and/or non-physiological parameters can also be determined, either from the data from the one or more sensors 210, or from other types of data.

The pressure sensor 212 outputs pressure data that can be stored in the memory device 204 and/or analyzed by the processor 202 of the control system 200. In some implementations, the pressure sensor 212 is an air pressure sensor (e.g., barometric pressure sensor) that generates sensor data indicative of the respiration (e.g., inhaling and/or exhaling) of the user of the respiratory therapy system 100 and/or ambient pressure. In such implementations, the pressure sensor 212 can be coupled to or integrated in the respiratory therapy device 110. The pressure sensor 212 can be, for example, a capacitive sensor, an electromagnetic sensor, a piezoelectric sensor, a strain-gauge sensor, an optical sensor, a potentiometric sensor, or any combination thereof.

The flow rate sensor 214 outputs flow rate data that can be stored in the memory device 204 and/or analyzed by the processor 202 of the control system 200. Examples of flow rate sensors (such as, for example, the flow rate sensor 214) are described in International Publication No. WO 2012/012835 and U.S. Pat. No. 10,328,219, both of which are hereby incorporated by reference herein in their entireties. In some implementations, the flow rate sensor 214 is used to determine an air flow rate from the respiratory therapy device 110, an air flow rate through the conduit 140, an air flow rate through the user interface 120, or any combination thereof. In such implementations, the flow rate sensor 214 can be coupled to or integrated in the respiratory therapy device 110, the user interface 120, or the conduit 140. The flow rate sensor 214 can be a mass flow rate sensor such as, for example, a rotary flow meter (e.g., Hall effect flow meters), a turbine flow meter, an orifice flow meter, an ultrasonic flow meter, a hot wire sensor, a vortex sensor, a membrane sensor, or any combination thereof. In some implementations, the flow rate sensor 214 is configured to measure a vent flow (e.g., intentional “leak”), an unintentional leak (e.g., mouth leak and/or mask leak), a patient flow (e.g., air into and/or out of lungs), or any combination thereof. In some implementations, the flow rate data can be analyzed to determine cardiogenic oscillations of the user. In some examples, the pressure sensor 212 can be used to determine a blood pressure of a user.

The temperature sensor 216 outputs temperature data that can be stored in the memory device 204 and/or analyzed by the processor 202 of the control system 200. In some implementations, the temperature sensor 216 generates temperatures data indicative of a core body temperature of the user 20, a skin temperature of the user 20, a temperature of the air flowing from the respiratory therapy device 110 and/or through the conduit 140, a temperature in the user interface 120, an ambient temperature, or any combination thereof. The temperature sensor 216 can be, for example, a thermocouple sensor, a thermistor sensor, a silicon band gap temperature sensor or semiconductor-based sensor, a resistance temperature detector, or any combination thereof.

The motion sensor 218 outputs motion data that can be stored in the memory device 204 and/or analyzed by the processor 202 of the control system 200. The motion sensor 218 can be used to detect movement of the user 20 during the sleep session, and/or detect movement of any of the components of the respiratory therapy system 100, such as the respiratory therapy device 110, the user interface 120, or the conduit 140. The motion sensor 218 can include one or more inertial sensors, such as accelerometers, gyroscopes, and magnetometers. In some implementations, the motion sensor 218 alternatively or additionally generates one or more signals representing bodily movement of the user, from which may be obtained a signal representing a sleep state of the user; for example, via a respiratory movement of the user. In some implementations, the motion data from the motion sensor 218 can be used in conjunction with additional data from another one of the sensors 210 to determine the sleep state of the user.

The microphone 220 outputs sound and/or audio data that can be stored in the memory device 204 and/or analyzed by the processor 202 of the control system 200. The audio data generated by the microphone 220 is reproducible as one or more sound(s) during a sleep session (e.g., sounds from the user 20). The audio data form the microphone 220 can also be used to identify (e.g., using the control system 200) an event experienced by the user during the sleep session, as described in further detail herein. The microphone 220 can be coupled to or integrated in the respiratory therapy device 110, the user interface 120, the conduit 140, or the user device 260. In some implementations, the system 10 includes a plurality of microphones (e.g., two or more microphones and/or an array of microphones with beamforming) such that sound data generated by each of the plurality of microphones can be used to discriminate the sound data generated by another of the plurality of microphones

The speaker 222 outputs sound waves that are audible to a user of the system 10 (e.g., the user 20 of FIG. 2). The speaker 222 can be used, for example, as an alarm clock or to play an alert or message to the user 20 (e.g., in response to an event). In some implementations, the speaker 222 can be used to communicate the audio data generated by the microphone 220 to the user. The speaker 222 can be coupled to or integrated in the respiratory therapy device 110, the user interface 120, the conduit 140, or the user device 260.

The microphone 220 and the speaker 222 can be used as separate devices. In some implementations, the microphone 220 and the speaker 222 can be combined into an acoustic sensor 224 (e.g., a SONAR sensor), as described in, for example, WO 2018/050913, WO 2020/104465, U.S. Pat. App. Pub. No. 2022/0007965, each of which is hereby incorporated by reference herein in its entirety. In such implementations, the speaker 222 generates or emits sound waves at a predetermined interval and the microphone 220 detects the reflections of the emitted sound waves from the speaker 222. The sound waves generated or emitted by the speaker 222 have a frequency that is not audible to the human ear (e.g., below 20 Hz or above around 18 kHz) so as not to disturb the sleep of the user 20 or the bed partner 30. Based at least in part on the data from the microphone 220 and/or the speaker 222, the control system 200 can determine a location of the user 20 and/or one or more of the sleep-related parameters described in herein such as, for example, a respiration signal, a respiration rate, an inspiration amplitude, an expiration amplitude, an inspiration-expiration ratio, a number of events per hour, a pattern of events, a sleep state, a sleep stage, pressure settings of the respiratory therapy device 110, or any combination thereof. In such a context, a sonar sensor may be understood to concern an active acoustic sensing, such as by generating and/or transmitting ultrasound and/or low frequency ultrasound sensing signals (e.g., in a frequency range of about 17-23 kHz, 18-22 kHz, or 17-18 kHz, for example), through the air.

In some implementations, the sensors 210 include (i) a first microphone that is the same as, or similar to, the microphone 220, and is integrated in the acoustic sensor 224 and (ii) a second microphone that is the same as, or similar to, the microphone 220, but is separate and distinct from the first microphone that is integrated in the acoustic sensor 224.

The RF transmitter 226 generates and/or emits radio waves having a predetermined frequency and/or a predetermined amplitude (e.g., within a high frequency band, within a low frequency band, long wave signals, short wave signals, etc.). The RF receiver 228 detects the reflections of the radio waves emitted from the RF transmitter 226, and this data can be analyzed by the control system 200 to determine a location of the user and/or one or more of the sleep-related parameters described herein. An RF receiver (either the RF receiver 226 and the RF transmitter 228 or another RF pair) can also be used for wireless communication between the control system 200, the respiratory therapy device 110, the one or more sensors 210, the user device 260, or any combination thereof. While the RF receiver 226 and RF transmitter 228 are shown as being separate and distinct elements in FIG. 1, in some implementations, the RF receiver 226 and RF transmitter 228 are combined as a part of an RF sensor 230 (e.g. a RADAR sensor). In some such implementations, the RF sensor 230 includes a control circuit. The format of the RF communication can be Wi-Fi, Bluetooth, or the like.

In some implementations, the RF sensor 230 is a part of a mesh system. One example of a mesh system is a Wi-Fi mesh system, which can include mesh nodes, mesh router(s), and mesh gateway(s), each of which can be mobile/movable or fixed. In such implementations, the Wi-Fi mesh system includes a Wi-Fi router and/or a Wi-Fi controller and one or more satellites (e.g., access points), each of which include an RF sensor that the is the same as, or similar to, the RF sensor 230. The Wi-Fi router and satellites continuously communicate with one another using Wi-Fi signals. The Wi-Fi mesh system can be used to generate motion data based on changes in the Wi-Fi signals (e.g., differences in received signal strength) between the router and the satellite(s) due to an object or person moving partially obstructing the signals. The motion data can be indicative of motion, breathing, heart rate, gait, falls, behavior, etc., or any combination thereof.

The camera 232 outputs image data reproducible as one or more images (e.g., still images, video images, thermal images, or any combination thereof) that can be stored in the memory device 204. The image data from the camera 232 can be used by the control system 200 to determine one or more of the sleep-related parameters described herein, such as, for example, one or more events (e.g., periodic limb movement or restless leg syndrome), a respiration signal, a respiration rate, an inspiration amplitude, an expiration amplitude, an inspiration-expiration ratio, a number of events per hour, a pattern of events, a sleep state, a sleep stage, or any combination thereof. Further, the image data from the camera 232 can be used to, for example, identify a location of the user, to determine chest movement of the user, to determine air flow of the mouth and/or nose of the user, to determine a time when the user enters the bed, and to determine a time when the user exits the bed. In some implementations, the camera 232 includes a wide angle lens or a fish eye lens.

The IR sensor 234 outputs infrared image data reproducible as one or more infrared images (e.g., still images, video images, or both) that can be stored in the memory device 204. The infrared data from the IR sensor 234 can be used to determine one or more sleep-related parameters during a sleep session, including a temperature of the user 20 and/or movement of the user 20. The IR sensor 234 can also be used in conjunction with the camera 232 when measuring the presence, location, and/or movement of the user 20. The IR sensor 234 can detect infrared light having a wavelength between about 700 nm and about 1 mm, for example, while the camera 232 can detect visible light having a wavelength between about 380 nm and about 740 nm.

The PPG sensor 236 outputs physiological data associated with the user 20 that can be used to determine one or more sleep-related parameters, such as, for example, a heart rate, a heart rate variability, a cardiac cycle, respiration rate, an inspiration amplitude, an expiration amplitude, an inspiration-expiration ratio, estimated blood pressure parameter(s), or any combination thereof. The PPG sensor 236 can be worn by the user 20, embedded in clothing and/or fabric that is worn by the user 20, embedded in and/or coupled to the user interface 120 and/or its associated headgear (e.g., straps, etc.), etc.

The ECG sensor 238 outputs physiological data associated with electrical activity of the heart of the user 20. In some implementations, the ECG sensor 238 includes one or more electrodes that are positioned on or around a portion of the user 20 during the sleep session. The physiological data from the ECG sensor 238 can be used, for example, to determine one or more of the sleep-related parameters described herein.

The EEG sensor 240 outputs physiological data associated with electrical activity of the brain of the user 20. In some implementations, the EEG sensor 240 includes one or more electrodes that are positioned on or around the scalp of the user 20 during the sleep session. The physiological data from the EEG sensor 240 can be used, for example, to determine a sleep state and/or a sleep stage of the user 20 at any given time during the sleep session. In some implementations, the EEG sensor 240 can be integrated in the user interface 120 and/or the associated headgear (e.g., straps, etc.).

The capacitive sensor 242, the force sensor 244, and the strain gauge sensor 246 output data that can be stored in the memory device 204 and used/analyzed by the control system 200 to determine, for example, one or more of the sleep-related parameters described herein. The EMG sensor 248 outputs physiological data associated with electrical activity produced by one or more muscles. The oxygen sensor 250 outputs oxygen data indicative of an oxygen concentration of gas (e.g., in the conduit 140 or at the user interface 120). The oxygen sensor 250 can be, for example, an ultrasonic oxygen sensor, an electrical oxygen sensor, a chemical oxygen sensor, an optical oxygen sensor, a pulse oximeter (e.g., SpO₂ sensor), or any combination thereof.

The analyte sensor 252 can be used to detect the presence of an analyte in the exhaled breath of the user 20. The data output by the analyte sensor 252 can be stored in the memory device 204 and used by the control system 200 to determine the identity and concentration of any analytes in the breath of the user. In some implementations, the analyte sensor 252 is positioned near a mouth of the user to detect analytes in breath exhaled from the user's mouth. For example, when the user interface 120 is a facial mask that covers the nose and mouth of the user, the analyte sensor 252 can be positioned within the facial mask to monitor the user's mouth breathing. In other implementations, such as when the user interface 120 is a nasal mask or a nasal pillow mask, the analyte sensor 252 can be positioned near the nose of the user to detect analytes in breath exhaled through the user's nose. In still other implementations, the analyte sensor 252 can be positioned near the user's mouth when the user interface 120 is a nasal mask or a nasal pillow mask. In this implementation, the analyte sensor 252 can be used to detect whether any air is inadvertently leaking from the user's mouth and/or the user interface 120. In some implementations, the analyte sensor 252 is a volatile organic compound (VOC) sensor that can be used to detect carbon-based chemicals or compounds. In some implementations, the analyte sensor 252 can also be used to detect whether the user is breathing through their nose or mouth. For example, if the data output by an analyte sensor 252 positioned near the mouth of the user or within the facial mask (e.g., in implementations where the user interface 120 is a facial mask) detects the presence of an analyte, the control system 200 can use this data as an indication that the user is breathing through their mouth.

The moisture sensor 254 outputs data that can be stored in the memory device 204 and used by the control system 200. The moisture sensor 254 can be used to detect moisture in various areas surrounding the user (e.g., inside the conduit 140 or the user interface 120, near the user's face, near the connection between the conduit 140 and the user interface 120, near the connection between the conduit 140 and the respiratory therapy device 110, etc.). Thus, in some implementations, the moisture sensor 254 can be coupled to or integrated in the user interface 120 or in the conduit 140 to monitor the humidity of the pressurized air from the respiratory therapy device 110. In other implementations, the moisture sensor 254 is placed near any area where moisture levels need to be monitored. The moisture sensor 254 can also be used to monitor the humidity of the ambient environment surrounding the user, for example, the air inside the bedroom.

The LiDAR sensor 256 can be used for depth sensing. This type of optical sensor (e.g., laser sensor) can be used to detect objects and build three dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having a LiDAR sensor 256 can measure and map an area extending 5 meters or more away from the sensor. The LiDAR data can be fused with point cloud data estimated by an electromagnetic RADAR sensor, for example. The LiDAR sensor(s) 256 can also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down, or falls down, for example. LiDAR may be used to form a 3D mesh representation of an environment. In a further use, for solid surfaces through which radio waves pass (e.g., radio-translucent materials), the LiDAR may reflect off such surfaces, thus allowing a classification of different type of obstacles.

In some implementations, the one or more sensors 210 also include a galvanic skin response (GSR) sensor, a blood flow sensor, a respiration sensor, a pulse sensor, a sphygmomanometer sensor, an oximetry sensor, a sonar sensor, a RADAR sensor, a blood glucose sensor, a color sensor, a pH sensor, an air quality sensor, a tilt sensor, a rain sensor, a soil moisture sensor, a water flow sensor, an alcohol sensor, or any combination thereof.

While shown separately in FIG. 1, any combination of the one or more sensors 210 can be integrated in and/or coupled to any one or more of the components of the system 10, including the respiratory therapy device 110, the user interface 120, the conduit 140, the humidifier 160, the control system 200, the user device 260, the activity tracker 270, or any combination thereof. For example, the microphone 220 and the speaker 222 can be integrated in and/or coupled to the user device 260 and the pressure sensor 212 and/or flow rate sensor 214 are integrated in and/or coupled to the respiratory therapy device 110. In some implementations, at least one of the one or more sensors 210 is not coupled to the respiratory therapy device 110, the control system 200, or the user device 260, and is positioned generally adjacent to the user 20 during the sleep session (e.g., positioned on or in contact with a portion of the user 20, worn by the user 20, coupled to or positioned on the nightstand, coupled to the mattress, coupled to the ceiling, etc.).

One or more of the respiratory therapy device 110, the user interface 120, the conduit 140, the display device 150, and the humidifier 160 can contain one or more sensors (e.g., a pressure sensor, a flow rate sensor, or more generally any of the other sensors 210 described herein). These one or more sensors can be used, for example, to measure the air pressure and/or flow rate of pressurized air supplied by the respiratory therapy device 110.

The data from the one or more sensors 210 can be analyzed (e.g., by the control system 200) to determine one or more sleep-related parameters, which can include a respiration signal, a respiration rate, a respiration pattern, an inspiration amplitude, an expiration amplitude, an inspiration-expiration ratio, an occurrence of one or more events, a number of events per hour, a pattern of events, a sleep state, an apnea-hypopnea index (AHI), or any combination thereof. The one or more events can include snoring, apneas, central apneas, obstructive apneas, mixed apneas, hypopneas, a mask leak, a cough, a restless leg, a sleeping disorder, choking, an increased heart rate, labored breathing, an asthma attack, an epileptic episode, a seizure, increased blood pressure, or any combination thereof. Many of these sleep-related parameters are physiological parameters, although some of the sleep-related parameters can be considered to be non-physiological parameters. Other types of physiological and non-physiological parameters can also be determined, either from the data from the one or more sensors 210, or from other types of data.

The user device 260 includes a display device 262. The user device 260 can be, for example, a mobile device such as a smart phone, a tablet, a gaming console, a smart watch, a laptop, or the like. Alternatively, the user device 260 can be an external sensing system, a television (e.g., a smart television) or another smart home device (e.g., a smart speaker(s) such as Google Home, Amazon Echo, Alexa etc.). In some implementations, the user device is a wearable device (e.g., a smart watch). The display device 262 is generally used to display image(s) including still images, video images, or both. In some implementations, the display device 262 acts as a human-machine interface (HMI) that includes a graphic user interface (GUI) configured to display the image(s) and an input interface. The display device 262 can be an LED display, an OLED display, an LCD display, or the like. The input interface can be, for example, a touchscreen or touch-sensitive substrate, a mouse, a keyboard, or any sensor system configured to sense inputs made by a human user interacting with the user device 260. In some implementations, one or more user devices can be used by and/or included in the system 10.

In some implementations, the system 10 also includes the activity tracker 270. The activity tracker 270 is generally used to aid in generating physiological data associated with the user. The activity tracker 270 can include one or more of the sensors 210 described herein, such as, for example, the motion sensor 218 (e.g., one or more accelerometers and/or gyroscopes), the PPG sensor 236, and/or the ECG sensor 238. The physiological data from the activity tracker 270 can be used to determine, for example, a number of steps, a distance traveled, a number of steps climbed, a duration of physical activity, a type of physical activity, an intensity of physical activity, time spent standing, a respiration rate, an average respiration rate, a resting respiration rate, a maximum he respiration art rate, a respiration rate variability, a heart rate, an average heart rate, a resting heart rate, a maximum heart rate, a heart rate variability, a number of calories burned, blood oxygen saturation, electrodermal activity (also known as skin conductance or galvanic skin response), or any combination thereof. In some implementations, the activity tracker 270 is coupled (e.g., electronically or physically) to the user device 260.

In some implementations, the activity tracker 270 is a wearable device that can be worn by the user, such as a smartwatch, a wristband, a ring, or a patch. For example, referring to FIG. 2, the activity tracker 270 is worn on a wrist of the user 20. The activity tracker 270 can also be coupled to or integrated a garment or clothing that is worn by the user. Alternatively still, the activity tracker 270 can also be coupled to or integrated in (e.g., within the same housing) the user device 260. More generally, the activity tracker 270 can be communicatively coupled with, or physically integrated in (e.g., within a housing), the control system 200, the memory device 204, the respiratory therapy system 100, and/or the user device 260.

In some implementations, the system 10 also includes the blood pressure device 280. The blood pressure device 280 is generally used to aid in generating cardiovascular data for determining one or more blood pressure measurements associated with the user 20. The blood pressure device 280 can include at least one of the one or more sensors 210 to measure, for example, a systolic blood pressure component and/or a diastolic blood pressure component.

In some implementations, the blood pressure device 280 is a sphygmomanometer including an inflatable cuff that can be worn by the user 20 and a pressure sensor (e.g., the pressure sensor 212 described herein). For example, in the example of FIG. 2, the blood pressure device 280 can be worn on an upper arm of the user 20. In such implementations where the blood pressure device 280 is a sphygmomanometer, the blood pressure device 280 also includes a pump (e.g., a manually operated bulb) for inflating the cuff. In some implementations, the blood pressure device 280 is coupled to the respiratory therapy device 110 of the respiratory therapy system 100, which in turn delivers pressurized air to inflate the cuff. More generally, the blood pressure device 280 can be communicatively coupled with, and/or physically integrated in (e.g., within a housing), the control system 200, the memory device 204, the respiratory therapy system 100, the user device 260, and/or the activity tracker 270.

In other implementations, the blood pressure device 280 is an ambulatory blood pressure monitor communicatively coupled to the respiratory therapy system 100. An ambulatory blood pressure monitor includes a portable recording device attached to a belt or strap worn by the user 20 and an inflatable cuff attached to the portable recording device and worn around an arm of the user 20. The ambulatory blood pressure monitor is configured to measure blood pressure between about every fifteen minutes to about thirty minutes over a 24-hour or a 48-hour period. The ambulatory blood pressure monitor may measure heart rate of the user 20 at the same time. These multiple readings are averaged over the 24-hour period. The ambulatory blood pressure monitor determines any changes in the measured blood pressure and heart rate of the user 20, as well as any distribution and/or trending patterns of the blood pressure and heart rate data during a sleeping period and an awakened period of the user 20. The measured data and statistics may then be communicated to the respiratory therapy system 100.

The blood pressure device 280 may be positioned external to the respiratory therapy system 100, coupled directly or indirectly to the user interface 120, coupled directly or indirectly to a headgear associated with the user interface 120, or inflatably coupled to or about a portion of the user 20. The blood pressure device 280 is generally used to aid in generating physiological data for determining one or more blood pressure measurements associated with a user, for example, a systolic blood pressure component and/or a diastolic blood pressure component. In some implementations, the blood pressure device 280 is a sphygmomanometer including an inflatable cuff that can be worn by a user and a pressure sensor (e.g., the pressure sensor 212 described herein).

In some implementations, the blood pressure device 280 is an invasive device which can continuously monitor arterial blood pressure of the user 20 and take an arterial blood sample on demand for analyzing gas of the arterial blood. In some other implementations, the blood pressure device 280 is a continuous blood pressure monitor, using a radio frequency sensor and capable of measuring blood pressure of the user 20 once very few seconds (e.g., every 3 seconds, every 5 seconds, every 7 seconds, etc.) The radio frequency sensor may use continuous wave, frequency-modulated continuous wave (FMCW with ramp chirp, triangle, sinewave), other schemes such as PSK, FSK etc., pulsed continuous wave, and/or spread in ultra wideband ranges (which may include spreading, PRN codes or impulse systems).

While the control system 200 and the memory device 204 are described and shown in FIG. 1 as being a separate and distinct component of the system 10, in some implementations, the control system 200 and/or the memory device 204 are integrated in the user device 260 and/or the respiratory therapy device 110. Alternatively, in some implementations, the control system 200 or a portion thereof (e.g., the processor 202) can be located in a cloud (e.g., integrated in a server, integrated in an Internet of Things (IoT) device, connected to the cloud, be subject to edge cloud processing, etc.), located in one or more servers (e.g., remote servers, local servers, etc., or any combination thereof.

While system 10 is shown as including all of the components described above, more or fewer components can be included in a system according to implementations of the present disclosure. For example, a first alternative system includes the control system 200, the memory device 204, and at least one of the one or more sensors 210 and does not include the respiratory therapy system 100. As another example, a second alternative system includes the control system 200, the memory device 204, at least one of the one or more sensors 210, and the user device 260. As yet another example, a third alternative system includes the control system 200, the memory device 204, the respiratory therapy system 100, at least one of the one or more sensors 210, and the user device 260. Thus, various systems can be formed using any portion or portions of the components shown and described herein and/or in combination with one or more other components.

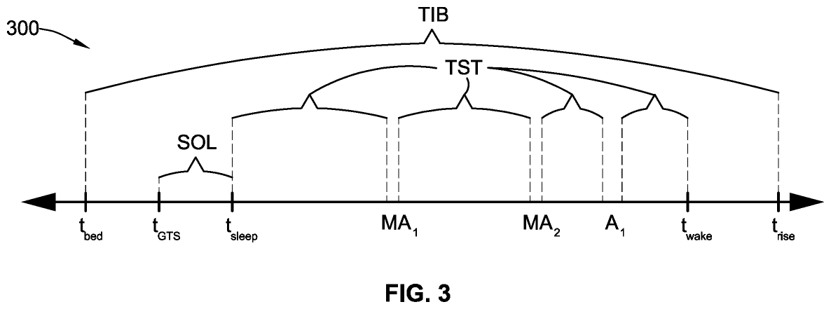

Referring now to FIG. 3, as used herein, a sleep session can be defined in a number of ways based at least in part on, for example, an initial start time and an end time. In some implementations, a sleep session is a duration where the user is asleep, that is, the sleep session has a start time and an end time, and during the sleep session, the user does not wake until the end time. That is, any period of the user being awake is not included in a sleep session. From this first definition of sleep session, if the user wakes ups and falls asleep multiple times in the same night, each of the sleep intervals separated by an awake interval is a sleep session.

Alternatively, in some implementations, a sleep session has a start time and an end time, and during the sleep session, the user can wake up, without the sleep session ending, so long as a continuous duration that the user is awake is below an awake duration threshold. The awake duration threshold can be defined as a percentage of a sleep session. The awake duration threshold can be, for example, about twenty percent of the sleep session, about fifteen percent of the sleep session duration, about ten percent of the sleep session duration, about five percent of the sleep session duration, about two percent of the sleep session duration, etc., or any other threshold percentage. In some implementations, the awake duration threshold is defined as a fixed amount of time, such as, for example, about one hour, about thirty minutes, about fifteen minutes, about ten minutes, about five minutes, about two minutes, etc., or any other amount of time.

In some implementations, a sleep session is defined as the entire time between the time in the evening at which the user first entered the bed, and the time the next morning when user last left the bed. Put another way, a sleep session can be defined as a period of time that begins on a first date (e.g., Monday, Jan. 6, 2020) at a first time (e.g., 10:00 PM), that can be referred to as the current evening, when the user first enters a bed with the intention of going to sleep (e.g., not if the user intends to first watch television or play with a smart phone before going to sleep, etc.), and ends on a second date (e.g., Tuesday, Jan. 7, 2020) at a second time (e.g., 7:00 AM), that can be referred to as the next morning, when the user first exits the bed with the intention of not going back to sleep that next morning.

In some implementations, the user can manually define the beginning of a sleep session and/or manually terminate a sleep session. For example, the user can select (e.g., by clicking or tapping) one or more user-selectable element that is displayed on the display device 262 of the user device 260 (FIG. 1) to manually initiate or terminate the sleep session.

FIG. 3 illustrates an exemplary timeline 300 for a sleep session. The timeline 300 includes an enter bed time (t_{bed}), a go-to-sleep time (t_{GTS}), an initial sleep time (tₛₗₑₑₚ), a first micro-awakening MA₁, a second micro-awakening MA₂, an awakening A, a wake-up time (t_{wake}), and a rising time (tᵣᵢₛₑ).

The enter bed time t_{bed} is associated with the time that the user initially enters the bed (e.g., bed 40 in FIG. 2) prior to falling asleep (e.g., when the user lies down or sits in the bed). The enter bed time t_{bed} can be identified based at least in part on a bed threshold duration to distinguish between times when the user enters the bed for sleep and when the user enters the bed for other reasons (e.g., to watch TV). For example, the bed threshold duration can be at least about 10 minutes, at least about 20 minutes, at least about 30 minutes, at least about 45 minutes, at least about 1 hour, at least about 2 hours, etc. While the enter bed time t_{bed} is described herein in reference to a bed, more generally, the enter time t_{bed} can refer to the time the user initially enters any location for sleeping (e.g., a couch, a chair, a sleeping bag, etc.).

The go-to-sleep time (GTS) is associated with the time that the user initially attempts to fall asleep after entering the bed (t_{bed}). For example, after entering the bed, the user may engage in one or more activities to wind down prior to trying to sleep (e.g., reading, watching TV, listening to music, using the user device 260, etc.). The initial sleep time (tₛₗₑₑₚ) is the time that the user initially falls asleep. For example, the initial sleep time (tₛₗₑₑₚ) can be the time that the user initially enters the first non-REM sleep stage.

The wake-up time t_{wake} is the time associated with the time when the user wakes up without going back to sleep (e.g., as opposed to the user waking up in the middle of the night and going back to sleep). The user may experience one of more unconscious microawakenings (e.g., microawakenings MA₁ and MA₂) having a short duration (e.g., 5 seconds, 10 seconds, 30 seconds, 1 minute, etc.) after initially falling asleep. In contrast to the wake-up time t_{wake}, the user goes back to sleep after each of the microawakenings MA₁ and MA₂. Similarly, the user may have one or more conscious awakenings (e.g., awakening A) after initially falling asleep (e.g., getting up to go to the bathroom, attending to children or pets, sleep walking, etc.). However, the user goes back to sleep after the awakening A. Thus, the wake-up time t_{wake} can be defined, for example, based at least in part on a wake threshold duration (e.g., the user is awake for at least 15 minutes, at least 20 minutes, at least 30 minutes, at least 1 hour, etc.).

Similarly, the rising time tᵣᵢₛₑ is associated with the time when the user exits the bed and stays out of the bed with the intent to end the sleep session (e.g., as opposed to the user getting up during the night to go to the bathroom, to attend to children or pets, sleep walking, etc.). In other words, the rising time tᵣᵢₛₑ is the time when the user last leaves the bed without returning to the bed until a next sleep session (e.g., the following evening). Thus, the rising time tᵣᵢₛₑ can be defined, for example, based at least in part on a rise threshold duration (e.g., the user has left the bed for at least 15 minutes, at least 20 minutes, at least 30 minutes, at least 1 hour, etc.). The enter bed time t_{bed} time for a second, subsequent sleep session can also be defined based at least in part on a rise threshold duration (e.g., the user has left the bed for at least 4 hours, at least 6 hours, at least 8 hours, at least 12 hours, etc.).

As described above, the user may wake up and get out of bed one more times during the night between the initial t_{bed} and the final tᵣᵢₛₑ. In some implementations, the final wake-up time t_{wale} and/or the final rising time tᵣᵢₛₑ that are identified or determined based at least in part on a predetermined threshold duration of time subsequent to an event (e.g., falling asleep or leaving the bed). Such a threshold duration can be customized for the user. For a standard user which goes to bed in the evening, then wakes up and goes out of bed in the morning any period (between the user waking up (t_{wake}) or raising up (tᵣᵢₛₑ), and the user either going to bed (t_{bed}), going to sleep (t_{GTS}) or falling asleep (tₛₗₑₑₚ) of between about 12 and about 18 hours can be used. For users that spend longer periods of time in bed, shorter threshold periods may be used (e.g., between about 8 hours and about 14 hours). The threshold period may be initially selected and/or later adjusted based at least in part on the system monitoring the user's sleep behavior.

The total time in bed (TIB) is the duration of time between the time enter bed time t_{bed} and the rising time tᵣᵢₛₑ. The total sleep time (TST) is associated with the duration between the initial sleep time and the wake-up time, excluding any conscious or unconscious awakenings and/or micro-awakenings therebetween. Generally, the total sleep time (TST) will be shorter than the total time in bed (TIB) (e.g., one minute short, ten minutes shorter, one hour shorter, etc.). For example, as shown in the timeline 300, the total sleep time (TST) spans between the initial sleep time tₛₗₑₑₚ and the wake-up time t_{wake}, but excludes the duration of the first micro-awakening MA₁, the second micro-awakening MA₂, and the awakening A. As shown, in this example, the total sleep time (TST) is shorter than the total time in bed (TIB).

In some implementations, the total sleep time (TST) can be defined as a persistent total sleep time (PTST). In such implementations, the persistent total sleep time excludes a predetermined initial portion or period of the first non-REM stage (e.g., light sleep stage). For example, the predetermined initial portion can be between about 30 seconds and about 20 minutes, between about 1 minute and about 10 minutes, between about 3 minutes and about 5 minutes, etc. The persistent total sleep time is a measure of sustained sleep, and smooths the sleep-wake hypnogram. For example, when the user is initially falling asleep, the user may be in the first non-REM stage for a very short time (e.g., about 30 seconds), then back into the wakefulness stage for a short period (e.g., one minute), and then goes back to the first non-REM stage. In this example, the persistent total sleep time excludes the first instance (e.g., about 30 seconds) of the first non-REM stage.

In some implementations, the sleep session is defined as starting at the enter bed time (t_{bed}) and ending at the rising time (tᵣᵢₛₑ), i.e., the sleep session is defined as the total time in bed (TIB). In some implementations, a sleep session is defined as starting at the initial sleep time (tₛₗₑₑₚ) and ending at the wake-up time (t_{wake}). In some implementations, the sleep session is defined as the total sleep time (TST). In some implementations, a sleep session is defined as starting at the go-to-sleep time (t_{GTS}) and ending at the wake-up time (t_{wake}). In some implementations, a sleep session is defined as starting at the go-to-sleep time (t_{GTS}) and ending at the rising time (tᵣᵢₛₑ). In some implementations, a sleep session is defined as starting at the enter bed time (t_{bed}) and ending at the wake-up time (t_{wake}). In some implementations, a sleep session is defined as starting at the initial sleep time (tₛₗₑₑₚ) and ending at the rising time (tᵣᵢₛₑ).

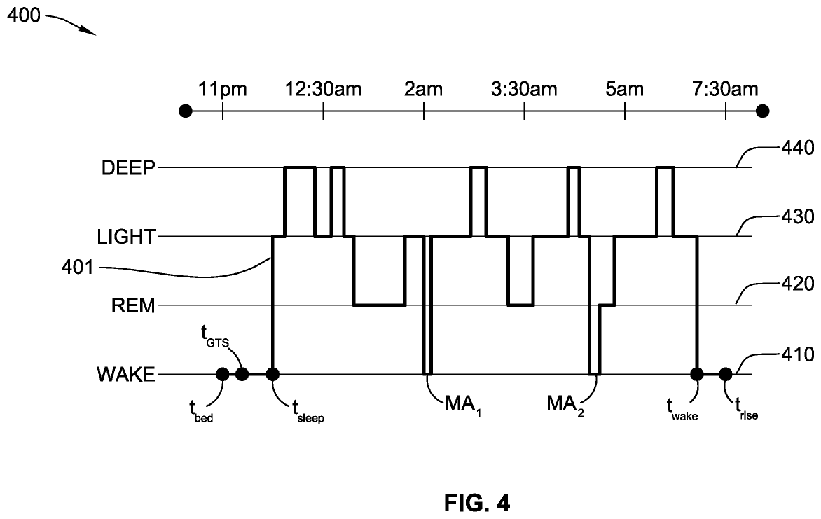

Referring to FIG. 4, an exemplary hypnogram 400 corresponding to the timeline 300 of FIG. 3, according to some implementations, is illustrated. As shown, the hypnogram 400 includes a sleep-wake signal 401, a wakefulness stage axis 410, a REM stage axis 420, a light sleep stage axis 430, and a deep sleep stage axis 440. The intersection between the sleep-wake signal 401 and one of the axes 410-440 is indicative of the sleep stage at any given time during the sleep session.

The sleep-wake signal 401 can be generated based at least in part on physiological data associated with the user (e.g., generated by one or more of the sensors 210 described herein). The sleep-wake signal can be indicative of one or more sleep stages, including wakefulness, relaxed wakefulness, microawakenings, a REM stage, a first non-REM stage, a second non-REM stage, a third non-REM stage, or any combination thereof. In some implementations, one or more of the first non-REM stage, the second non-REM stage, and the third non-REM stage can be grouped together and categorized as a light sleep stage or a deep sleep stage. For example, the light sleep stage can include the first non-REM stage and the deep sleep stage can include the second non-REM stage and the third non-REM stage. While the hypnogram 400 is shown in FIG. 4 as including the light sleep stage axis 430 and the deep sleep stage axis 440, in some implementations, the hypnogram 400 can include an axis for each of the first non-REM stage, the second non-REM stage, and the third non-REM stage. In other implementations, the sleep-wake signal can also be indicative of a respiration signal, a respiration rate, an inspiration amplitude, an expiration amplitude, an inspiration-expiration amplitude ratio, an inspiration-expiration duration ratio, a number of events per hour, a pattern of events, or any combination thereof. Information describing the sleep-wake signal can be stored in the memory device 204.

The hypnogram 400 can be used to determine one or more sleep-related parameters, such as, for example, a sleep onset latency (SOL), wake-after-sleep onset (WASO), a sleep efficiency (SE), a sleep fragmentation index, sleep blocks, or any combination thereof.

The sleep onset latency (SOL) is defined as the time between the go-to-sleep time (t_{GTS}) and the initial sleep time (tₛₗₑₑₚ). In other words, the sleep onset latency is indicative of the time that it took the user to actually fall asleep after initially attempting to fall asleep. In some implementations, the sleep onset latency is defined as a persistent sleep onset latency (PSOL). The persistent sleep onset latency differs from the sleep onset latency in that the persistent sleep onset latency is defined as the duration time between the go-to-sleep time and a predetermined amount of sustained sleep. In some implementations, the predetermined amount of sustained sleep can include, for example, at least 10 minutes of sleep within the second non-REM stage, the third non-REM stage, and/or the REM stage with no more than 2 minutes of wakefulness, the first non-REM stage, and/or movement therebetween. In other words, the persistent sleep onset latency requires up to, for example, 8 minutes of sustained sleep within the second non-REM stage, the third non-REM stage, and/or the REM stage. In other implementations, the predetermined amount of sustained sleep can include at least 10 minutes of sleep within the first non-REM stage, the second non-REM stage, the third non-REM stage, and/or the REM stage subsequent to the initial sleep time. In such implementations, the predetermined amount of sustained sleep can exclude any micro-awakenings (e.g., a ten second micro-awakening does not restart the 10-minute period).

The wake-after-sleep onset (WASO) is associated with the total duration of time that the user is awake between the initial sleep time and the wake-up time. Thus, the wake-after-sleep onset includes short and micro-awakenings during the sleep session (e.g., the micro-awakenings MA₁ and MA₂ shown in FIG. 4), whether conscious or unconscious. In some implementations, the wake-after-sleep onset (WASO) is defined as a persistent wake-after-sleep onset (PWASO) that only includes the total durations of awakenings having a predetermined length (e.g., greater than 10 seconds, greater than 30 seconds, greater than 60 seconds, greater than about 5 minutes, greater than about 10 minutes, etc.)

The sleep efficiency (SE) is determined as a ratio of the total time in bed (TIB) and the total sleep time (TST). For example, if the total time in bed is 8 hours and the total sleep time is 7.5 hours, the sleep efficiency for that sleep session is 93.75%. The sleep efficiency is indicative of the sleep hygiene of the user. For example, if the user enters the bed and spends time engaged in other activities (e.g., watching TV) before sleep, the sleep efficiency will be reduced (e.g., the user is penalized). In some implementations, the sleep efficiency (SE) can be calculated based at least in part on the total time in bed (TIB) and the total time that the user is attempting to sleep. In such implementations, the total time that the user is attempting to sleep is defined as the duration between the go-to-sleep (GTS) time and the rising time described herein. For example, if the total sleep time is 8 hours (e.g., between 11 PM and 7 AM), the go-to-sleep time is 10:45 PM, and the rising time is 7:15 AM, in such implementations, the sleep efficiency parameter is calculated as about 94%.

The fragmentation index is determined based at least in part on the number of awakenings during the sleep session. For example, if the user had two micro-awakenings (e.g., micro-awakening MA₁ and micro-awakening MA₂ shown in FIG. 4), the fragmentation index can be expressed as 2. In some implementations, the fragmentation index is scaled between a predetermined range of integers (e.g., between 0 and 10).

The sleep blocks are associated with a transition between any stage of sleep (e.g., the first non-REM stage, the second non-REM stage, the third non-REM stage, and/or the REM) and the wakefulness stage. The sleep blocks can be calculated at a resolution of, for example, 30 seconds.

In some implementations, the systems and methods described herein can include generating or analyzing a hypnogram including a sleep-wake signal to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof based at least in part on the sleep-wake signal of a hypnogram.

In other implementations, one or more of the sensors 210 can be used to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof, which in turn define the sleep session. For example, the enter bed time t_{bed} can be determined based at least in part on, for example, data generated by the motion sensor 218, the microphone 220, the camera 232, or any combination thereof. The go-to-sleep time can be determined based at least in part on, for example, data from the motion sensor 218 (e.g., data indicative of no movement by the user), data from the camera 232 (e.g., data indicative of no movement by the user and/or that the user has turned off the lights), data from the microphone 220 (e.g., data indicative of the using turning off a TV), data from the user device 260 (e.g., data indicative of the user no longer using the user device 260), data from the pressure sensor 212 and/or the flow rate sensor 214 (e.g., data indicative of the user turning on the respiratory therapy device 110, data indicative of the user donning the user interface 120, etc.), or any combination thereof.

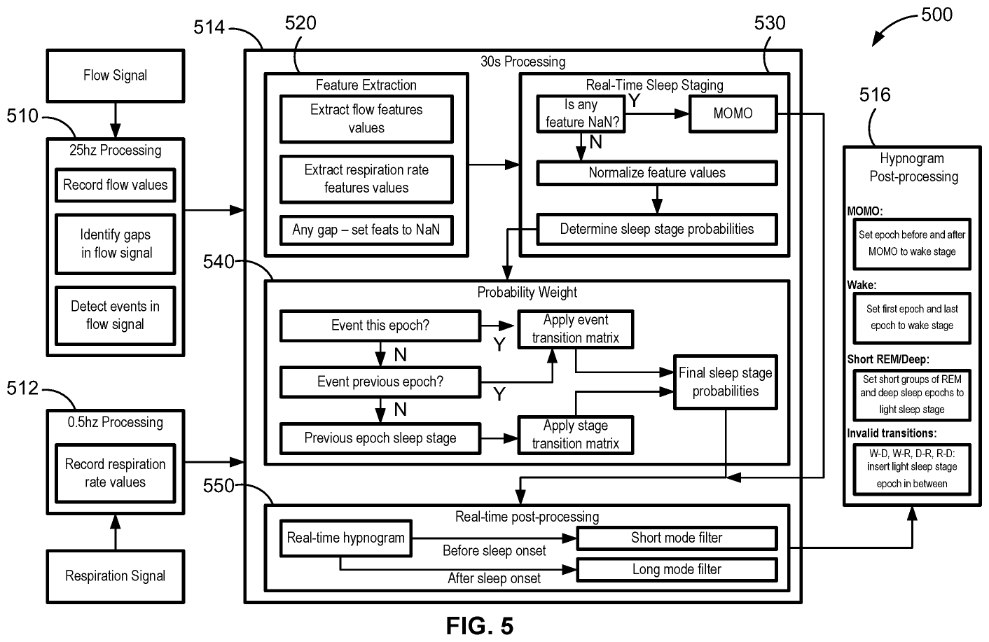

FIG. 5 illustrates a block diagram of an example algorithm 500 for determining various sleep stages of a user (such us user 20) during a sleep session. Various portions of algorithm 500 can be implemented using system 10 or components of system 10. For example, any data utilized in algorithm 500 could be generated by the one or more sensors 210.

The algorithm 500 divides the sleep session into a plurality of individual segments referred to as epochs. The algorithm 500 can determine the sleep stage that the user was in during each epoch, and create a hypnogram showing the various sleep stages of the user throughout the sleep session. In some implementations, the possible sleep stages are a wake stage, a light sleep stage, a deep sleep stage, and a REM sleep stage. Other implementations may include more or less sleep stages. For example, other implementations may separate the light sleep stage into the N1 stage and the N2 stage, and the deep sleep stage into the N3 and N4 stages. In these implementations, the possible sleep stages are a wake stage, an N1 stage, an N2 stage, and N3 stage, and N4 stage, and a REM sleep stage. In still other implementations, only the light sleep stage is separated into multiple stages. Thus, the possible sleep stages in these implementations are a wake stage, an N1 stage, an N2 stage, a deep sleep stage, and a REM sleep stage. In further implementations, only the deep sleep stage is separated into multiple stages. Thus, the possible sleep stages in these implementations are a wake stage, a light sleep stage, an N3 stage, an N3 stage, and a REM sleep stage. In even further implementations, the potential sleep stages include only a wake stage and a sleep stage.

Algorithm 500 can take as input a variety of data generated by the system 10 during the sleep session, including data generated by the one or more sensors 210. The data can include a flow signal representative of the flow of pressurized air from the respiratory therapy device 110 to the user interface 120 via the conduit 140, as the user breathes during the sleep session. Generally, the flow signal is a measure of the volume of flow per unit time. The physiological data can also include a respiration signal representative of the user's respiration. Generally, the respiration signal is a measure of the amplitude of the user's respiration. Temporal data can also be generated by system 10 (e.g., data related to the time duration of the sleep session, data related to which epoch of the sleep session is the current epoch, etc.).

As shown in FIG. 5, the algorithm includes three separate processing blocks 510, 512, and 514 that can operate at different rates. In the illustrated implementation, block 510 operates at 25 Hz (e.g., 25 cycles per second), and every cycle extracts an individual flow value from the flow signal. An individual flow value generally refers to a distinct flow rate, e.g., n Liters/second (L/s). However, block 510 could operate at different rates in other implementations. The flow values (e.g., the values of the flow signal at certain times) can then be stored for later use, for example in a memory device (such as memory device 204). In certain instances, the user interface that is worn by the user may fall off, or may be inadvertently or deliberately removed from the user's face. In these instances, there may be a gap in the flow signal, and thus no individual flow value will be stored for that cycle. Finally, as block 510 is monitoring the flow signal and recording flow values, block 510 also determines for every cycle whether the flow signal indicates the occurrence of a respiratory event, such as an apnea, a hypopnea, a RERA, a flow limitation event, etc. In some implementations, block 510 determines only whether an apnea, a hypopnea, and/or a RERA occurred during the epoch. In other implementations, other events can be identified, such as snoring, an intentional user interface leak, an unintentional user interface leak, a mouth leak, a cough, a restless leg, a sleeping disorder, choking, an increased heart rate, labored breathing, an asthma attack, an epileptic episode, a seizure, increased blood pressure, hyperventilation, or any combination thereof. In some implementations, block 510 (or other blocks of algorithm 500) can also determine if other events occurred that may impact the user's sleep during the sleep session, such as an alarm going off or other loud noises occurring.

In the illustrated implementation, block 512 operates at 0.5 Hz (e.g., one cycle every two seconds), and every cycle extracts a respiration rate value from the respiration signal. However, block 512 could operate at different rates in other implementations, such as between about 0.1 Hz and about 2.0 Hz. Generally, a respiration rate value must be extracted at least one for every epoch. Thus, if the length of the epochs is 30 seconds, block 512 could operate with a minimum frequency of about 0.03 Hz

$$( e . g . , \frac{1}{30} Hz ) .$$

The respiration rate values (e.g., the values of the respiration rate signal at certain times) can then be stored for later use, for example in a memory device (such as memory device 204). The flow values, the gaps in the flow values, and the event occurrences are sent from block 510 to block 514. Similarly, the respiration rate values are sent from block 512 to block 514.

In some implementations, the flow signal and the respiration signal are separate signals that can be input into the algorithm 500. However, in other implementations, respiration rate values can be derived from the flow signal. In these implementations, the respiration signal can be obtained from the flow signal, and then the respiration rate values are obtained from the respiration signal. In a first example of these other implementations, the flow signal itself may be input into both block 510 and block 512, and block 512 can derive the respiration rate values from the flow signal. In a second example of these other implementations, the flow signal can be analyzed (for example by the control system 200 of the system 10) to obtain the respiration rate signal, and then respiration rate signal is input into the block 512. In a third example of these other implementations, the flow signal can be analyzed (for example by the control system 200 of the system 10) to obtain the respiration rate values, which can then be input into the block 512 (which may then record one of the respiration rate values every cycle). In further implementations, respiration rate values are obtained directly from the flow signal (e.g., without first obtaining a respiration signal from the flow signal), and can then be input into block 512. In additional implementations, individual flow values and individual respiration rate values can be input directly into block 510 and block 512. Block 510 can record one of the flow values every cycle, and block 512 can record one of the respiration rate values every cycle.

Block 514 analyzes the data received from block 510 and block 512 and determines, for every individual epoch in the sleep session, which sleep stage that the user is in for that epoch. Generally, block 514 analyzes the data in real-time, and thus every time an epoch ends, the data from that epoch is analyzed by block 514 to determine which sleep stage the user was in during that epoch. In the illustrated implementation, each epoch lasts for about 30 seconds, and thus block 514 analyzes the data in 30-second increments (e.g., increments that span about 30 seconds). In other implementations however, different lengths for the epochs can be used. For example, the length of the epochs could be about 1 second, about 5 seconds, about 10 seconds, about 20 seconds, about 1 minute, about 2 minutes, about 5 minutes, or about 10 minutes. The length of the epochs can also be set to correspond to a specific number of breaths, such as 1 breath, 2 breaths, 5 breaths, etc. The length can be determined based on the user's average respiration rate, or an average respiration rate for a population to which the user belongs.

Block 514 is formed from four sub-blocks. Sub-block 520 extracts respiratory-related features for each epoch from the data sent by block 510 and block 512. Sub-block 530 generates sleep stage probabilities for each epoch based at least in part on the extracted features. Sub-block 540 adjusts the sleep stage probabilities for each epoch based at least in part on events occurring during that epoch, events occurring during one or more prior epochs, and/or the sleep stage probabilities of the one or more prior epochs. Sub-block 550 performs real-time post-processing on the sleep stage probabilities for each epoch based at least in part the sleep stage probabilities of surrounding epochs, and the position of the epoch within the sleep session. In the illustrated implementation, block 514 operates in real-time, and thus the functions of block 514 are performed immediately after all of the data for an epoch has been stored and sent to block 514. As used herein, the term “current epoch” refers to the epoch that has just finished, and is currently being analyzed at block 514 to determine which sleep stage the user was in during that epoch. However, in other implementations, block 514 could operate after the sleep session has been completed. In these implementations though, the term “current epoch” will still refer to the epoch that is currently being analyzed at block 514.

For each epoch, sub-block 520 analyzes both the flow data from block 510 and the respiration data from block 512 to extract one or more features associated with the epoch, which can then be used to determine which sleep stage the user is in during the epoch. These features can then be stored for future use by other sub-blocks or blocks. The extracted features can generally be grouped into one of three different categories. The first category of features includes features associated with the flow of pressurized air from the respiratory therapy device 110 to the user interface 120 during the epoch. The second category of features includes features associated with the user 20's respiration rate during the sleep session. The third category includes features associated with the temporal location of the epoch within the sleep session.

The flow-related features can be extracted by analyzing the flow data captured by block 510. A first flow-related feature that can be extracted is the maximum flow across the current epoch and/or one or more prior epochs. Generally, the wake stage and the light sleep stage have higher maximum flow values than the deep sleep stage and the REM sleep stage. Moreover, large maximum flow values (which could be caused by, for example, the user gasping or hyperventilating) can be indicative of the user being in the wake stage, for example. In the illustrated implementation, a single epoch will have 750 stored flow values, because block 510 extracts a flow value 25 times per second, and each epoch lasts for 30 seconds. Sub-block 520 analyzes all stored flow values for the current epoch and at least one prior epoch, and thus selects a single flow value from at least 1,500 stored flow values. In one implementation, the first feature is the maximum flow value over the current epoch and the last two epochs, in which case sub-block 520 would select the maximum flow value out of 2,250 stored flow values. In some implementations, this feature is the maximum flow value over the current epoch and at least one prior epoch. The at least one prior epoch can include one prior epoch, two prior epochs, three prior epochs, four prior epochs, five prior epochs, or ten prior epochs. However, in other implementations, this feature may be the maximum flow value across only the current epoch.

A second flow-related feature that can be captured is the spread of the flow values within the current epoch (and/or one or more prior epochs). The spread of the flow values measures how clustered together the flow values for the current epoch are, and can be expressed as describing the range within which a threshold percentage of the flow values fall. In some implementations, the threshold percentage is about 68%, but other percentages can also be used. The spread of the flow values will generally be larger for the wake stage and the light sleep stage, as compared to the deep sleep stage and the REM sleep stage. The spread of the flow values can be extracted by calculating the standard deviation of the flow values according to the following equation:

$$spread = \sqrt{\frac{{Σ ( {Q}_{i} - μ )}^{2}}{N}} .$$

Here, Qᵢ is the i^{th} flow value within the current epoch; μ is the average flow value for the epoch; and N is the number of flow values within the current epoch. In the illustrated implementations, N=750, meaning that the Qᵢ values will range from Q₁ (the 1^{st} flow value within the epoch) to Q₇₅₀ (the 750^{th} flow value within the epoch). In some implementations, the spread of the flow values can be determined across multiple epochs, which can include the current epoch and one or more prior epochs, or simply two or more prior epochs.

A third flow-related feature is the stability of the spread of the flow values over the current epoch and/or one or more prior epochs. This feature is a measure of how stable the flow spreads are for multiple epochs (and not how stable the flow values themselves are). In general, as the user progresses to deep sleep, the flow spreads being to stabilize more, and do not vary as much on an epoch-to-epoch basis. The stability of the spread of the flow values is calculated by taking the standard deviation of the standard deviation of flow values across the current epoch and one or more prior epochs, according to the following equation:

$$stability of spread = \sqrt{\frac{{Σ ( {Q}_{{std}_{i}} - μ )}^{2}}{N}} .$$

Here, Q_{std}_{ᵢ} is the spread of the flow values of the i^{th} epoch; μ is the average flow value spread across all i epochs; and N is the number of flow value spreads (e.g., N=i). In some implementations, the stability of the spread of flow values is measured across the current epoch and two prior epochs. In these implementations, N=3, there will be three different flow value spreads, and the average flow value spread will be the average of those three flow value spreads. In other implementations, the stability of the spread of the flow values can be determined across the current epoch and one prior epoch, the current epoch and three or more prior epochs, or only two or more prior epochs.

A fourth flow-related feature that can be captured is the skew of the flow values for the current epoch (and/or one or more prior epochs). The skew of the flow values within an epoch measures the distribution of the flow values is around the average flow value for the epoch. The skew measures how asymmetrical that the distribution of the flow values are relative to the average flow value. The skew measures both the direction of the asymmetry, as well as the magnitude of the asymmetry. A flow value distribution that is perfectly symmetrical about the average flow value would have a skew value of zero. A positive skew over the epoch means that the user emphasized inhaling over exhaling during the epoch (e.g., more flow occurred during inhaling than exhaling). A negative skew over the epoch means that the user emphasized exhaling over inhaling during the epoch (e.g., more flow occurred during exhaling than inhaling). Generally, the wake stage and the light sleep stage will have more positive skew values, while the deep sleep stage and the REM sleep stage will have more negative skew values, as there is more emphasis on the exhale in the deep sleep and REM sleep stages. In some implementations, the skew of the flow value is measured as the Fisher-Pearson skewness, and is determined according to the following equation:

$$Skew = \sqrt{\frac{{Σ ( {Q}_{i} - μ )}^{3}}{{σ}^{3}}} .$$

Here, Qᵢ is the i^{th} flow value within the current epoch; μ is the average flow value for the epoch, and σ is the standard deviation of the flow values within the current epoch (which is itself the second flow-related feature).

A fifth flow-related feature that can be captured is a smoothed version of the skew of the flow values. This feature is a smoothed-out version of the fourth flow-related feature, and is less susceptible to artifacts in the data for the epoch (for example, artifacts caused by events). The smoothed skew can be calculated simply by taking the median skew value over the desired epoch or epochs. In some implementations, the smoothed skew is the median skew value across the current epoch and the prior nine epochs. In other implementations, the smoothed skew is the median skew value across the current epoch and one to five prior epochs.

A sixth flow-related feature is the stability of the flow volume across the current epoch and/or one or more prior epochs. The flow volume is simply a measure of how much air that the user is breathing during the epoch, and will generally become more stable as the user enters the deep sleep stage and the REM sleep stage. The flow volume can be calculated by adding all of the individual flow values for the epoch. The stability of the flow volume can then be calculated by taking the standard deviation of the flow volumes for the current epoch and the one or more prior epochs, according to the following equation:

$$flow volume stability = \sqrt{\frac{{Σ ( {Q}_{{absVol}_{i}} - μ )}^{2}}{N}} .$$

Here, Q_{absVol}_{ᵢ} is the flow volume for the i^{th} epoch; μ is the average flow volume across all i epochs; and N is the number of calculated flow volumes (e.g., N=i). In some implementations, the stability of the flow volumes is measured across the current epoch and nine prior epochs. In these implementations, N=10, there will be ten different flow volumes, and the average flow volume will be the average of those ten flow volumes. In other implementations, the stability of the flow volumes is measured across the current epoch and nineteen prior epochs. In these implementations, N=20, there will be twenty different flow volumes, and the average flow volume will be the average of those twenty flow volumes. In additional implementations, the stability of the flow volume across these two epochs spans (N=10 and N=20) are extracted as two separate features. In further implementations, the stability of the flow volume can be calculated across the current epoch and any number of prior epochs.

In some implementations, additional features can be extracted from the flow values for the current epoch and/or one or more prior epochs. These additional features can include a time ratio of inspiration to expiration for the epoch, a volume ratio of inspiration to expiration for the current epoch, or both. In some implementations, features related to the flow of pressurized air supplied by the respiratory therapy device can be based on the pressure parameters of the pressurized air (e.g., can be extracted from the pressure signal of the sleep session). For example, the features could include a maximum pressure of the pressurized air during the current epoch and/or one or more prior epochs, an average pressure of the pressurized air during the current epoch and/or one or more prior epochs, a spread of pressure values of the pressurized air during the current epoch and/or one or more prior epochs, a stability of the spread of pressure values of the pressurized air during the current epoch and/or one or more prior epochs, other pressure-related features, and any combinations thereof. Other features related to the flow of pressurized air and based on the pressure parameters could include a skew of pressure values of the pressurized air during the current epoch and/or one or more prior epochs, a smoothed skew of pressure values of the pressurized air during the current epoch and/or one or more prior epochs, or a combination thereof.

The respiration rate-related features can be extracted by analyzing the respiration data captured by block 512. A first respiration rate-related feature that can be extracted is the average respiration rate for the current epoch. Generally, the user's respiration rate will slow down as the user moves to deep sleep stages, and will speed up as the user moves to REM sleep stages and wake stages. This feature can be calculated by adding the value of each individual respiration rate for the current epoch that is captured by block 512, and then diving the sum by the number of respiration rate samples for the current epoch. In the illustrated implementation, because each epoch is 30 seconds long and block 512 operates at a frequency of 0.5 Hz, the average respiration rate for the current epoch is calculated over fifteen respiration rate values.

A second respiration rate-related feature that can be captured is the variability of the average respiration rate values across the current epoch and one or more prior epochs. The variability of the average respiration rate values measures how the average respiration rate values vary over the course of multiple epochs. The average respiration rate values will generally be less variable during deep sleep stages (and in some cases light sleep stages), but will be more variable during REM sleep stages. The variability of the average respiration rate values can be extracted by calculating the standard deviation of the average respiration rate values according to the following equation:

$$variability = \sqrt{\frac{{Σ ( {rr}_{{avg}_{i}} - μ )}^{2}}{N}} .$$

Here, rr_{avg}_{ᵢ} is the average respiration rate of the i^{th} epoch; μ is the average of all of the average respiration rate values for the current epoch and the one or more prior epochs; and N is the number of average respiration rate values. In some implementations, the variability of the average respiration rate values is measured across the current epoch and nine prior epochs. In these implementations, N=10, there will be ten different average respiration rate values, and the average will be the average of those ten average respiration rate values. In other implementations, the variability of the average respiration rate values is measured across the current epoch and nineteen prior epochs. In these implementations, N=20, there will be twenty different average respiration rate values, and the average will be the average of those twenty average respiration rate values. In additional implementations, the variability of the average respiration rate values across these two epochs spans (N=10 and N=20) are extracted as two separate features. In further implementations, the variability of the average respiration rate values can be calculated across the current epoch and any number of prior epochs.

In some implementations, the features are associated with additional characteristics of the user's respiration, or a different characteristic of the user's respiration instead of the user's respiration rate. For example, the features could be associated with respiration rate variability, inspiration amplitude, expiration amplitude, inspiration-expiration amplitude ratio, inspiration-expiration duration ratio, number of events per hour, pattern of events, pressure settings of the respiratory therapy device 110, or any combination thereof.

The third category includes features associated with the temporal location of the epoch within the sleep session. Generally, if the epoch is closer to the beginning of the sleep session, it is more likely that the sleep stage will be either a wake stage or a deep sleep stage. If the epoch is further away from the beginning of the sleep session, it is more likely that the sleep stage will be either a deep sleep stage or a light sleep stage. If the epoch is closer to the end of the sleep session, it is more likely that the sleep stage will be a REM sleep stage. A first temporal feature is the count of the epoch within the sleep session. For example, if the current epoch is the fifth epoch within the sleep session, the value of the first temporal feature is five. If the current epoch is the 267^{th} epoch with the sleep session, the value of the first temporal feature is 267. A second temporal feature is the 20^{th} root of the count of the epoch within the sleep session. This second temporal feature is similar to the first temporal feature, but takes the 20^{th} root of the count, instead of the count itself. Referring back to the example, if the current epoch is the fifth epoch within the sleep session, the value of the second temporal feature is

$$\sqrt[20]{5} = 1.083798 .$$

If the current epoch is the 267^{th} epoch within the sleep session, the value of the second temporal feature is

$$\sqrt[20]{267} = 1.322286 .$$

Other temporal features can also be extracted that use different roots of the epoch count, that transform the epoch count in a different manner (e.g., the log or the square of the epoch count), or that indicate the location of the epoch within the sleep session in a different manner.

In some implementations, the epoch count begins when the sleep session begins. In other implementations, the epoch count begins when the user initially falls asleep during the sleep session. In other implementations, the epoch may reset every time that the user wakes up. Generally, algorithm 500 can include an epoch counter that is incremented every time an epoch is complete (e.g., every time 30 seconds worth of flow data and respiration data is captured and stored).

Sub-block 520 also analyzes the data for each epoch to determine if there is any gap in the data associated with the user interface not being worn by the user during at least a portion of the current epoch. If there is a gap in the data for an epoch such that the value of any feature cannot be extracted and stored for the epoch, sub-block 520 stores the feature values for the epoch as “Not a Number” (also referred to as “NaN”).

Sub-block 530 performs real-time sleep staging based on the stored values of any one or more of the features discussed herein. Sub-block 530 uses a trained model (such as a trained machine learning algorithm) to generate a plurality of sleep stage probabilities. The model can be trained using training data, which includes data from prior sleep sessions that has been correlated with confirmed sleep stages in the sleep session. The training data can be generated during sleep studies performed in hospitals and other healthcare facilities, for example. The confirmed sleep stages may be determined using, for example, polysomnography. Each of the sleep stage probabilities is a measure of the probability that the user was in a respective one of the potential sleep stages during the current epoch.

The sub-block 530 first determines if the value of any of the features for the current epoch (the values being stored by sub-block 520) is set as Not a Number. If there is a feature for the current epoch that is set as Not a Number, the current epoch is designated as “Mask-Off Mask-On,” or “MOMO.” The MOMO designation for the current epoch is then sent to sub-block 550 for real-time post-processing.

If none of the features for the current epoch are set as Not a Number, sub-block 530 proceeds to standardize the values of each of the extracted features. Standardizing the values of the features places the feature values on the same scale and allows for the values to be more easily compared when determining which sleep stage the individual is in during the current epoch. The feature values can be standardized to all be between −1 and +1, −2 and +2, and other scales. To standardize the feature values, the mean value for the feature is subtracted from the actual value, and then divided by the standard deviation of that feature value. In some implementations, the mean and standard deviation of the feature values are generated from the training data that was used to train the model. In other implementations, the mean and standard deviation of the feature values are generated using previously-obtained data from the user's current sleep session.

In some implementations, sub-block 530 normalizes the values instead of standardizing the values. In these implementations, sub-block 530 can divide all of the values by the maximum value, such that all of the normalized values are set to between 0 and 1. In some implementations, prior to standardizing or normalizing the feature values, sub-block 530 first discards or corrects any features that have outlier values as compared to some baseline value or baseline range for that feature. A minimum and maximum value for each feature can be established, such that there is a pre-defined range of possible values for each feature. Then, any feature having a value outside of the pre-defined range of possible values for that feature (e.g., a value greater than the maximum value or less than the minimum value) can be discarded, or be amended to the maximum value (if greater than the maximum value) or the minimum value (if less than the minimum value). In one example, the baseline value or baseline range is determined from training data used to train the model.

After the feature values have been standardized, sub-block 530 can input the standardized feature values into the trained model. The trained model processes the standardized feature values, and outputs sleep stage probabilities. In some implementations, four sleep stage probabilities are outputted: the probability that the user was in the wake stage during the current epoch, the probability that the user was in the light sleep stage during the current epoch, the probability that the user was in the deep sleep stage during the current epoch, and the probability that the user was in the REM sleep stage during the current epoch. Generally, the sum of these four sleep stage probabilities is equal to 100%.

In some implementations, the model is a multilayer perceptron model, which is a class of feedforward artificial neural networks. In other implementations, the model can be a logistic regression model, a decision tree, a naïve Bayes Gaussian model, a rigid classification model, a linear discriminant model, a quadratic model, a support-vector machine (SVM) model, or any other classifier that can be used to predict an output based on a set of inputs. A variety of different controllable variables can affect the performance of the model, including which features are input into the model, how the features are transformed prior to being input into the model, how many sleep stages the model can classify the epoch as, which epochs are used to train the model, and others.

To train the model to properly classify the epochs, a number of different parameters can be adjusted. These parameters include the learning rate (which affects how much various weights are adjusted after each training step), the regularization constant (which affects how well the model is fit to the data), how many internal layers the model includes between the input layer and the outputs, how many nodes each layer has, and the maximum number of iterations that the model performs. In one implementation, the model was trained using a learning rate of 0.001, a regularization constant of 0.001, and a maximum number of iterations of 400. The model being trained included one internal layer having twelve nodes.

In some implementations, the model can include one input layer, one hidden layer, and one output layer. The input layer includes a number of input nodes that corresponds to the number of feature values that are input into the model. Thus, each input node corresponds to the value of a single feature. Each input node passes its feature value to each of the hidden nodes. Similar to the input layer, the hidden layer includes a number of hidden nodes that corresponds to the number of feature values that are input into the model. At each hidden node, the following calculation is performed:

$${z}_{1} = Σ ( {a}_{{0}_{i}} * {w}_{{1}_{i}} ) + {b}_{1}$$

Here, aₒ_{ᵢ} is the value of the i^{th} feature (e.g., the inputs to the hidden nodes received from the input nodes), w₁_{ᵢ} is a pre-determined weighting value for the i^{th} feature at the hidden node, b₁ is a pre-determined bias value for the hidden node, and z₁ is the intermediate output of the hidden node. At each hidden node, the weighting value can be different for each different input (e.g., feature value from one of the input nodes). Further, each hidden node can have different weighting values and bias values as compared to other hidden nodes. Thus, at a given hidden node, the value of each feature (from the input nodes) is weighted, the sum of that total for each feature is determined, and the bias value is added to that sum. Then, for each hidden node, the value of an activation function is determined using the intermediate output z₁ for that respective hidden node. In some implementations, the activation function for the hidden nodes is a Rectified Linear Unit (ReLU) given by a₁_{ᵢ}=relu (z₁)=max(z₁, 0). The ReLU function thus outputs the input z₁ if the input is positive, and outputs 0 if the input z₁ is 0 or negative. Thus, each hidden node produced a single output a₁.

The output layer includes an output node for each different potential sleep stage probability. Generally, each output node corresponds to one of the sleep stage probabilities. The output of each hidden node is passed to each output node of the output layer. At each output node, the following calculation is performed:

$${z}_{2} = Σ ( {a}_{{1}_{i}} * {w}_{{2}_{i}} ) + {b}_{2}$$

Here, a₁_{ᵢ} is the output of the i^{th} hidden node, w₂ is a pre-determined weighting value for the i^{th} output node, b₂ is a pre-determined bias value for the i^{th} output node, and z₂ is the intermediate output of the output node. At each output node, the weighting value can be different for each different input (e.g., the output of each hidden node). Further, each output node can have different weighting values and bias values as compared to other output nodes. Thus, at a given output node, the output of each hidden node is weighted, the sum of that total for each hidden node is determined, and the bias value is added to that sum. Then, for each output node, the value of an activation function is determined using the intermediate output z₂ for that respective output node. In some implementations, the activation function for the output nodes is a sigmoid function given by

$${a}_{2} = sigmoid ( {z}_{2} ) = \frac{1}{1 + {e}^{- {z}_{2}}} .$$

The output a₂ of each output node is the sleep stage probability for the sleep stage that the output corresponds to.

Generally, the model can have any number of layers, and any number of nodes at each layer, including the input layer. Thus, in some implementations the input layer may include one input node for each feature, but in other implementations, the input layer includes more input nodes than features, or less input nodes than features. Any combination of layers and nodes can be used, so long as the output layer includes a number of output nodes equal to the number of potential sleep stages that the user may be in during the epoch (e.g., the number of output nodes is equal to the number of different sleep stage probabilities that the model is used to generate).

The sleep stage probabilities include the probability that the user was in the wake stage (P(w)) during the current epoch, the probability that the user was in the light sleep stage (P(l)) during the current epoch, the probability that the user was in the deep sleep stage (P(d)) during the current epoch, and the probability that the user was in the REM sleep stage (P(r)) during the current epoch. In other implementations, the model may be configured to generate more than or less than four sleep stage probabilities. Generally, each of the sleep stage probabilities is a decimal between 0 and 1. However, the sleep stage probabilities could also be expressed as a percent between 0 and 100.

These sleep stage probabilities can then be sent to sub-block 540, which applies weights to the sleep stage probabilities (e.g., multiplies the sleep stage probabilities by a set of coefficients). Sub-block 540 first determines whether an event occurred during the current epoch, based on the data generated by block 510. If an event did occur during the current epoch, the sleep stage probabilities are passed to an event transition matrix, where the sleep stage probabilities are weighted by multiplying the sleep stage probabilities by a first set of coefficients. In some implementations, the value of each coefficient in the first set of coefficients is a decimal number between 0 and 1. In other implementations however, one or more of the coefficients in the first set of coefficients could have a value that is greater than 1. Generally, the first set of coefficients include a respective coefficient for each combination of sleep stage and event type. In some implementations, there are three distinct events that can affect the sleep stage probabilities. These events are apneas, hypopneas, and RERAs. Thus, in implementations where there are four sleep stage probabilities for the current epoch, the first set of coefficients includes twelve total coefficients—one for each combination of four sleep stages and three event types. The twelve coefficients are shown here:

$$[ \begin{matrix}a : w & a : l & a : d & a : r \\ h : w & h : l & h : d & h : r \\ re : w & re : l & re : d & re : r\end{matrix} ] .$$

The first letter of each coefficient refers to the type of event experienced during the current epoch: a=apnea, h=hypopnea, and re=RERA. The second letter of each coefficient refers to the sleep stage probability that the coefficient is to be applied to: w=wake stage, l=light sleep stage, d=deep sleep stage, and r=REM sleep stage. Thus, for example, a:w is the coefficient that the wake sleep stage probability for the current epoch is multiplied by if an apnea occurred during the current epoch; h:d is the coefficient that the deep sleep stage probability for the current epoch is multiplied by if a hypopnea occurred during the current epoch; and re:r is the coefficient that the REM sleep stage probability for the current epoch is multiplied by if a RERA occurred during the current epoch. In some cases, multiple events may occur during a single epoch. In these cases, algorithm 500 includes a priority order for determining which coefficients of the first set of coefficients are to be used. In some implementations, the priority order is apnea, hypopnea, and RERA. In these implementations, if an apnea is detected along with either a hypopnea and a RERA, the coefficients corresponding to the apnea event will be applied to the sleep stage probabilities. Similarly, if a hypopnea and a RERA are detected, the coefficients corresponding to the hypopnea event will be applied to the sleep stage probabilities. Finally, the coefficients corresponding to the RERA event will only be applied to the sleep stage probabilities if only RERA events are detected. In other implementations, different priority orders can be used.

While the event transition matrix is shown as having twelve coefficients, algorithm 500 can be used to identify any number of sleep stages and events, and thus the event transition matrix can generally include any number of coefficients, as may be required. Once the event transition matrix has been applied and each of the sleep stage probabilities has been adjusted by multiplying by its appropriate coefficient in the first set of coefficients, these final sleep stage probabilities are passed to sub-block 550.

If sub-block 540 determines that no event occurred during the current epoch, sub-block 540 then looks to the immediately prior epoch to determine if an event occurred during that epoch. If an event did occur during the prior epoch, the sleep stage probabilities are again passed to the event transition matrix, where the first set of coefficients are applied to the sleep stage probabilities as discussed. The final sleep stage probabilities are then passed to sub-block 550.

In the illustrated implementation, the same first set of coefficients is applied to the sleep stage probabilities regardless of whether the event occurred in the current epoch or the prior epoch. However, in other implementations, different sets of coefficients can be applied based on whether the event occurred in the current epoch or the prior epoch. Both sets of coefficients will generally have the same types of coefficients, e.g., one coefficient applied to the wake stage probability if the event was an apnea, one coefficient applied to the light sleep stage probability if the event was a hypopnea, etc. However, the coefficients themselves will generally have different values, so that the adjusted sleep stage probabilities will have different values. In further implementations however, an entirely different set of coefficients (with a different number of coefficients) can be applied if the event occurred during the prior epoch as compared to the current epoch.

In some implementations, the event transition matrix can be used to adjust the sleep stage probabilities if the previous epoch was designated as Mask-On Mask-Off Generally, if the previous epoch was a Mask-On Mask-Off epoch, the algorithm 500 is configured to set the current epoch to the wake stage. Thus, the event transition matrix can include coefficients that when applied, set the sleep stage probability for the wake stage to 100% (or 1), and the sleep stage probabilities of the light sleep stage, the deep sleep stage, and the REM sleep stage to 0% (or 0).

In some implementations, sub-block 540 may also determine whether any events occurred in a subsequent epoch, and adjust the sleep stage probabilities for the current epoch based on the events in the subsequent epoch. The subsequent epoch generally occurs immediately after the current epoch, but could also occur later in the sleep session as well. For example, if an apnea event occurs during a subsequent epoch, it is more likely that the previous epoch was a REM sleep stage, since apnea events are more likely to occur during REM sleep stages. The sleep stage probabilities for the current epoch could then be adjusted due to the detected apnea event to increase the REM sleep stage probability. In these implementations, the algorithm 500 will generally operate on the data from the sleep session in a delayed fashion, or retroactively after the sleep session has been completed. Thus, when sub-block 540 of block 514 operates on the current epoch, block 510 has already operated on the subsequent epoch to identify events experienced during the subsequent epoch. As such, the sub-block 540 can use any events experienced during the subsequent epoch to adjust the sleep stage probabilities of the current epoch. In these implementations, if an event occurred during the subsequent epoch, sub-block 540 will pass the sleep stage probabilities for the current epoch to the event transition matrix, where the sleep stage probabilities are weighted by multiplying the sleep stage probabilities by the first set of coefficients.

In some implementations, the transition matrix may apply the same coefficients to the sleep stage probabilities regardless of whether the event occurred in the current epoch, the prior epoch, or the subsequent epoch, where the coefficients are specific to the type of event that occurred. In other implementations, the transition matrix may include different coefficients based on whether the event occurred in the current epoch, the prior epoch, or the subsequent epoch.

However, if sub-block 540 determines that no event occurred during the immediately prior epoch either, sub-block 540 looks at what the determined sleep stage was for the immediately prior epoch (e.g., the sleep stage with the highest probability), and then passes the sleep stage probabilities to a stage transition matrix. The stage transition matrix is similar to the event transition matrix, and includes a second set of coefficients to be applied to the sleep stage probabilities. However, the stage transition matrix includes a respective coefficient for each combination of prior epoch sleep stage and current epoch sleep stage. Thus, in implementations where there are four possible sleep stages used to classify the epoch, the stage transition matrix includes sixteen coefficients, which are shown here:

$$[ \begin{matrix}w : w & w : l & w : d & w : r \\ l : w & l : l & l : d & l : r \\ d : w & d : l & d : d & d : r \\ r : w & r : l & r : d & r : r\end{matrix} ] .$$

The first letter of each coefficient refers to the sleep stage of the immediately prior epoch (e.g., the sleep stage having the highest probability), and the second letter of each coefficient refers to the sleep stage of the current epoch. Thus, for example, w:w is the coefficient that the wake stage probability for the current epoch is multiplied by if the wake sleep stage had the highest probability in the immediately prior epoch; d:l is the coefficient that the light sleep stage probability for the current epoch is multiplied by if the deep sleep stage had the highest probability in the immediately prior epoch; and l:r is the coefficient that the REM sleep stage probability for the current epoch is multiplied by if the light sleep stage had the highest probability in the immediately prior epoch. While the stage transition matrix is shown as having sixteen coefficients, algorithm 500 can be used to identify any number of sleep stages, and thus the stage transition matrix can generally include any number of coefficients, as may be required. Once the stage transition matrix has been applied and each of the sleep stage probabilities has been adjusted by multiplying by its appropriate coefficient in the second set of coefficients, these final sleep stage probabilities are passed to sub-block 550.

In some implementations, sub-block 540 can also adjust the sleep stage probabilities for the current epoch based on the sleep stage of the subsequent epoch (immediately after the current epoch or later in the sleep session). Similar to the adjustment based on events occurring during the subsequent epoch, the algorithm 500 will generally operate on the data from the sleep session in a delayed fashion, or retroactively after the sleep session has been completed. Thus, in these implementations, when sub-block 540 operates on the current epoch, sub-block 530 will have at least determined the initial sleep stage probabilities for the subsequent epoch (e.g., the sleep stage probabilities before the probabilities are weighted by the event transition matrix and/or the stage transition matrix). As such, the sub-block 540 can use the initial sleep stage probabilities for the subsequent epoch to adjust the sleep stage probabilities for the current epoch. In some cases, sub-block 540 may treat the sleep stage with the highest initial sleep stage probability for the subsequent epoch as the determined sleep stage for the subsequent epoch. The sub-block 540 can pass the sleep stage probabilities to the stage transition matrix, where the sleep stage probabilities are weighted by multiplying the sleep stage probabilities by the second set of coefficients.

In some implementations, the stage transition matrix applies the same coefficients to the sleep stage probabilities for the current epoch regardless of whether sub-block 540 is comparing to the past epoch or the subsequent epoch. Thus, if the highest sleep stage probability in both the prior epoch and the subsequent epoch is the same sleep stage, then the same coefficients are applied to the sleep stage probabilities for the current epoch. However, in other implementations, different coefficients are used depending on whether sub-block 540 is comparing to the prior epoch or the subsequent epoch. Thus, even if the same sleep stage has the highest probability for both the prior epoch and the subsequent epoch, different coefficients would be applied to the sleep stage probabilities for the current epoch, depending on whether the prior epoch or the subsequent epoch was being used.

The algorithm 500 may look to the prior epoch or the subsequent epoch based on different circumstances. For example, the algorithm 500 could select between the prior epoch and the subsequent epochs) based on the initial sleep stage probabilities for the current epoch. If the initial sleep stage probabilities have a first value/range of values, sub-block 540 can determine what sleep stage the prior epoch was, and then apply the corresponding coefficients from the stage transition matrix. If the initial sleep stage probabilities have a second value/range of values, sub-block 540 can determine what sleep stage the subsequent epoch was, and then apply the corresponding coefficients from the stage transition matrix. In cases where the prior epoch and the subsequent epoch have the same sleep stage and the same coefficients for the same sleep stages, it does not matter whether the prior or subsequent epoch is used. However, in cases where the prior epoch and the subsequent epoch have the same sleep stage, but different coefficients for the same sleep stage, the algorithm 500 can again select between the prior epoch and the subsequent epoch based on different circumstances, such as the initial sleep stage probabilities of the current epoch. The algorithm 500 could use other factors as well, such as the location of the current epoch within the sleep session (e.g., earlier or later within the sleep session), the flow of the pressurized air, the pressure of the pressurized air, physiological parameters of the user (such as respiration-related parameters), and other factors.

Sub-block 550 generates a hypnogram from the sleep stage probabilities for each epoch, and then performs real-time filtering on the hypnogram. The hypnogram (which may be similar to hypnogram 400 of FIG. 4) is generated by selecting one of the potential sleep stages as the actual sleep stage for each epoch. Generally, the sleep stage with the highest adjusted sleep stage probability (e.g., the highest sleep stage probability after being weighted by the first set of coefficients or the second set of coefficients) is selected as the sleep stage for the current epoch.

In some implementations, this hypnogram generated by sub-block 550 is the final output of the example algorithm, and can be used to analyzed the user's sleep session, as well as other tasks. In other implementations however, sub-block 550 applies filters to the hypnogram in order to smooth the transitions between epochs. When filtered, the hypnogram is smoother and includes more concrete steps when transitioning between different sleep stages, instead of including multiple smaller transitions back and forth between different sleep stages. In the illustrated implementations, sub-block 550 applies a short-mode filter to epochs occurring before sleep onset, and a long-mode filter to epochs occurring after sleep onset. For both filters, sub-block 550 looks at a group of consecutive epochs, determines which sleep stage is the most common for that group of epochs, and modifies the epochs within the group so that each epoch in the group is set to the most common sleep stage. For example, if sub-block 550 looked at a group of five epochs whose sleep stages were a wake stage, a light sleep stage, a light sleep stage, a wake stage, and a light sleep stage, sub-block 550 would determine that the light sleep stage is the most common sleep stage within the group of epochs, and then modify the wake stage epochs to instead be set as the light sleep stage. Sub-block 550 would then move to the next group of epochs. In some implementations, each group of epochs contains entirely distinct epochs, such that no epoch is common to multiple groups and will be filtered twice. In other implementations however, there may be overlap between the epochs in adjacent groups.

The number of epochs within the group depends on whether sub-block 550 is applying the short mode filter (before sleep onset) or the long mode filter (after sleep onset). Generally, the number of epochs within each group when applying the short mode filter is less than the number of epochs within each group when applying the long mode filter. In one example, the number of epochs within each group when applying the short mode filter is five epochs, and the number of epochs within each group when applying the long mode filter is seven or eight epochs. However, other numbers of epochs can be used for the short mode filter and the long mode filter as well. If within a given group of epochs there is a tie, the sleep stages of those epochs can be set according to a pre-determined ranking of the different potential sleep stages. In some implementations, this ranking is, in order, the wake stage, the light sleep stage, the deep sleep stage, and finally the REM sleep stage.

In some implementations, sub-block 550 transitions from the short mode filter to the long mode filter once a given group of epochs is set to one of the sleep stages. Generally, this is the light sleep stage, but it could be the deep sleep stage or the REM sleep stage. Thus, the last group of epochs where sub-block 550 applies the short mode filter is the first group of epochs where the most common epoch is the light sleep stage, the deep sleep stage, or the REM sleep stage.

Thus, the inputs to block 514 of algorithm 500 include the flow values, the respiration rate values, and the event occurrences that are generated at block 510 and block 512. The output of block 514 is a hypnogram that designates as the sleep stage for each epoch, the sleep stage having the highest probability for that epoch, as determined by sub-block 530, sub-block 540, and sub-block 550.

Block 516 can operate on the hypnogram for a final set of post-processing steps. In some implementations, block 516 operates on the hypnogram once the sleep session has finished. However, in other implementations, block 516 can also operate on the hypnogram in real-time as sub-block 550 outputs the selected sleep stage for each epoch. Block 516 analyzes the hypnogram for potential errors in the epochs, based on the sleep stages for each epoch.

In a first example, algorithm 500 assumes that a certain number of epochs prior to or subsequent to a Mask-On Mask-Off epoch should be set to the wake stage. Thus, block 516 identifies epochs marked as Mask-On Mask-Off, and if the predetermined number of prior epochs and the predetermined number of subsequent epochs are set to anything other than the wake stage, block 516 changes the sleep stage for those epochs to the wake stage. In this example however, prior sleep stages or subsequent sleep stages within the predetermined number from a Mask-On Mask-Off epoch that are also designated as a Mask-On Mask-Off epoch are not altered. Thus, if there is a string of consecutive epochs designated as Mask-On Mask-Off epochs, algorithm 500 will only look at the predetermined number of epochs prior to the first Mask-On Mask-Off epoch in the string, and the predetermined number of epochs subsequent to the last Mask-On Mask-Off epoch in the string. The predetermined number of prior and subsequent epochs can be the same or different, and can be any number of epochs. In one implementations, algorithm 500 checks the two epochs prior to the Mask-On Mask-Off epochs, and the five epochs subsequent to Mask-On Mask-Off epoch.

In a second example, algorithm 500 assumes that the first epoch of the sleep session and the last epoch of the sleep session should both be set to the wake stage. If either of these epochs are not set to the wage stage, block 516 changes these epochs to the wake stage.

In a third example, algorithm 500 assumes that both deep sleep stages and REM sleep stages last for at least a predetermined minimum amount of time. Thus, block 516 identifies any group of one or more epochs set to either the deep sleep stage or the REM sleep stage, and modifies those epochs if they do not meet the predetermined minimum amount of time. In some implementations, the minimum amount of time for REM sleep stages and deep sleep stages are different from each other. Generally, the minimum amount of time for REM sleep stages is less than the minimum amount of time for deep sleep stages, but in some cases the minimum amount of time for deep sleep stages may be less. In one example of these implementations, the minimum amount of time for REM sleep stages is one minute (which is equal to two epochs if the epochs are 30 seconds long), and the minimum amount of time for deep sleep stages is three minutes (which is equal to six epochs if the epochs are 30 seconds long). In other implementations, the minimum amount of time for REM sleep stages is the same as the minimum amount of time for deep sleep stages. In some implementations, the epochs within groups of both deep sleep stages and REM sleep stages that do not last for the minimum amount of time are all changed to the light sleep stage. In other implementations however, the epochs within these groups could be changed to other sleep stages.

In a fourth example, algorithm 500 assumes that there are certain transitions between sleep stages that are not allowed. If block 516 detects two adjacent epochs with one of the non-allowed transition, block 516 can correct the hypnogram. A first non-allowed transition is from a wake stage directly to a deep sleep stage. If block 516 detects this transition, block 516 can correct the hypnogram by changing the wake stage epoch to a light sleep stage epoch, changing the deep sleep stage epoch to a light sleep stage epoch, or inserting a light sleep stage epoch between the wake stage epoch and the deep sleep stage epoch. A second non-allowed transition is from a wake stage directly to a REM sleep stage. If block 516 detects this transition, block 516 can correct the hypnogram by changing the wake stage epoch to a light sleep stage epoch, changing the REM sleep stage epoch to a light sleep stage epoch, or inserting a light sleep stage epoch between the wake stage epoch and the REM sleep stage epoch.

A third non-allowed transition is from a deep sleep stage directly to a REM sleep stage. If block 516 detects this transition, block 516 can correct the hypnogram by changing the deep sleep stage epoch to a light sleep stage epoch, changing the REM sleep stage epoch to a light sleep stage epoch, or inserting a light sleep stage epoch between the deep sleep stage epoch and the REM sleep stage epoch. A fourth non-allowed transition is from a REM sleep stage directly to a deep sleep stage. If block 516 detects this transition, block 516 can correct the hypnogram by changing the REM sleep epoch to a light sleep stage epoch, changing the deep sleep stage epoch to a light sleep stage epoch, or inserting a light sleep stage epoch between the REM sleep stage epoch and the deep sleep stage epoch. After block 516 finishes correcting the initial hypnogram, the final hypnogram can be used for any further analysis of the sleep session that may be desired. In some implementations, the user (or a third party) can review the hypnogram and provide feedback. The feedback can then be used to adjust the operation of any of the block 510, block 512, block 514, and block 516.

In various implementations, the algorithm 500 may include more or less than the blocks and sub-blocks shown in FIG. 5, and the various functions performed by the blocks and sub-blocks can be performed in different orders and/or at different times. In a first example, the sleep-staging performed by sub-block 530 can be done after the entire sleep session has been completed, instead of in real-time. The feature values can thus be standardized based on their values across the entire sleep session. In a second example, values of any number of the features can be standardized (or normalized) in real-time, or after the sleep session has been completed. In a third example, the sleep stage probabilities are not weighted in real-time, but instead are altered after the sleep session has been completed, and are altered based on the sleep stage probabilities for all of the epochs of the sleep session. Generally, any portion of algorithm 500 can be performed in real-time during the sleep session, or after the sleep session has been completed. Thus, the various functions of algorithm 500 can all be performed in real-time during the sleep session, can all be performed after the sleep session has been completed, or can be performed in a combination of real-time during the sleep session and after the sleep session has been completed.

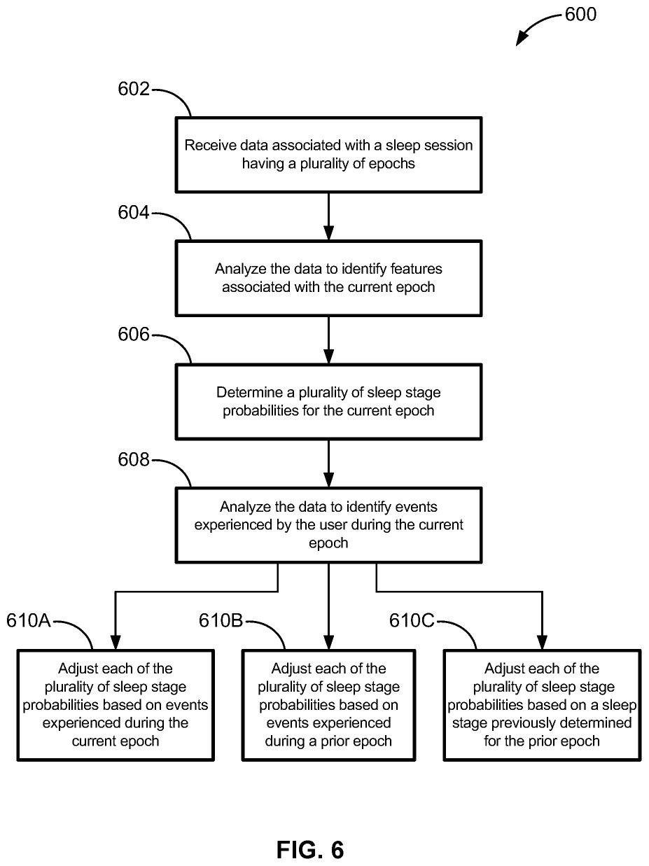

FIG. 6 illustrates a method 600 for determining a sleep stage of an individual. Generally, a control system (such as the control system 200 of the system 10) is configured to carry out the various steps of method 600. A memory device (such as the memory device 204 of the system 10) can be used to store any type of data utilized in the steps of method 600 (or other methods). Method 600 is a specific implementation of the algorithm 500, which can be used with a variety of different methods.

Step 602 of method 600 includes receiving data associated with a sleep session that has a plurality of epochs. In some implementations, the data includes flow data representative of the flow of pressurized air from the respiratory therapy device 110 to the user interface 120 via the conduit 140, and respiration data representative of the user's respiration. In some implementations, the flow data and the respiration data are generated by sensors associated with the respiratory therapy device 110 and/or the respiratory therapy system 100 (such as sensors 210). Generally, step 602 corresponds to block 510 and block 512 of algorithm 500 receiving the flow signal and the respiration signal and recording the individual flow values and respiration rate values.

At step 604 of method 600, the data is analyzed to identify features associated with the current epoch. Generally, step 604 corresponds to sub-block 520 of algorithm 500. As noted, the sleep session can be divided into a plurality of epochs. The epochs can be any suitable length, such as 30 seconds long. At step 604, any flow data representative of the flow of pressurized air during the epoch is analyzed, along with any respiration data representative of the user's respiration rate during the epoch. A number of different features can be identified. In some implementations, the features include one or more features that are associated with the flow of the pressurized air, such as (i) a maximum flow value across the current epoch and one or more prior epochs, (ii) a flow skew of the current epoch, (iii) a median flow skew across the current epoch and one or more prior epochs, (iv) a standard deviation of flow values for the current epoch, (v) a standard deviation of the standard deviation of flow values for the current epoch and one or more prior epochs, (vi) standard deviation of the flow volume for the current epoch and one or more of the prior epochs (vii) a time ratio of inspiration to expiration for the current epoch, (viii) a ratio of inspiration volume to expiration volume for the current epoch, or (ix) any combination of (i)-(viii).

In some implementations, the features include one or more features that are associated with the respiration rate of the user, such as the average respiration rate across the current epoch, a standard deviation of the respiration rate across the current epoch and one or more prior epochs, or both. In some implementations, the features can include at least one feature associated with a temporal property of the current epoch. Generally, the temporal property is some measurement of where the epoch is located within the sleep session. For example, the temporal property can be the number of the current epoch within the current sleep session or the n^{th} root of the number of the current epoch within the current sleep session. In some implementations, n=20, and the temporal property is the 20^{th} root of the number of the current epoch within the current sleep session.

Step 606 of method 600 includes determining a plurality of sleep stage probabilities for the current epoch. In some implementations, the user may be in one of a plurality of potential sleep stages during the current epoch. These sleep stages can include a wake stage, a light sleep stage, a deep sleep stage, and a REM sleep stage. In some implementations, the potential sleep stages may include more sleep stages or fewer sleep stages. In any of these implementations, step 606 includes determining the probability that the user was in each of the potential sleep stages during the current epoch. In some implementations, the determination of the sleep stage probabilities is based at least in part on at least one flow-related feature, at least one respiratory rate-related feature, and at least one feature associated with a temporal property of the current epoch. In other implementations however, the determination of the sleep stage probabilities can be based at least in part on any combination of flow-related, respiratory rate-related, and temporal features, and/or other features.

In some implementations, the determination of the sleep stage probabilities is performed by a trained machine learning algorithm that has been trained using a set of training data. The training data could, for example, be obtained from sleep studies where the sleep stages are determined and verified using, for example, the “gold standard” polysomnography technique, and with corresponding flow data and respiration data. In some implementations, the trained machine learning algorithm is a multilayer perceptron model, which is a class of feedforward artificial neural networks. In other implementations, other types of machine learning algorithms can be used, as well as other techniques for determining sleep stage probabilities. Step 606 generally corresponds to sub-block 530 of algorithm 500.

In some implementations, method 600 can include discarding any features for the current epoch that have outlier values. For example, if the average respiration rate for the epoch is determined to be unrealistically high, that feature can be discarded, and will not be used to determine the sleep stage probabilities for the current epoch. In some implementations, the feature values can be compared to baseline values or baseline ranges for that feature. In some implementations, the baseline values or ranges are generated from the training data. In other implementations, the baseline values or ranges are generated using previously-obtained data from the user's current sleep session and/or one or more prior sleep sessions. In some implementations, the values of the features can also be standardized to all be on the same scale (such as between −1 and +1, −2 and +2, etc.).

Step 608 of method 600 includes analyzing the data to identify events experienced by the user during the current epoch. The flow data and/or the respiration data can indicate if the user suffered from any events during the current epoch. In some implementations, the events include respiratory-related events such as apneas, hypopneas, and/or RERAs.

After features from the current epoch have been extracted and events occurring during the current epoch have been identified, the plurality of sleep stage probabilities can be adjusted in steps 610A, 610B, or 610C, based on events occurring during the current epoch, events occurring during the prior epoch, or the determined sleep stage of the prior epoch. Steps 610A, 610B, and 610C generally correspond to sub-block 540 of algorithm 500.

If one or more events did occur during the current epoch, method 600 proceeds from step 608 to step 610A, where each of the sleep stage probabilities is adjusted based at least in part on the one or more events that occurred during the current epoch.

In some implementations, adjusting the plurality of sleep stage probabilities in step 610A includes applying a first set of coefficients to the sleep stage probabilities. The first set of coefficients includes a plurality of coefficients corresponding to the potential sleep stages and potential events. Each coefficient in the first set of coefficients is associated with both (i) a single one of the potential sleep stages, and (ii) a single one of the potential events that can occur during the sleep session. The first set of coefficients includes a respective coefficient for each distinct combination of sleep stages and events. In some implementations, the potential sleep stages include a wake stage, a light sleep stage, a deep sleep stage, and a REM sleep stage; and the events that may occur during the sleep session and be detected include an apnea, a hypopnea, and a RERA. Thus, in these implementations, the first set of coefficients would include twelve distinct coefficients. Generally, the first set of coefficients can include as many coefficients as are necessary, based on the number of potential sleep stages and the number of events that may occur during the sleep session.

Generally, step 606 of method 600 will include determining a sleep stage probability for each potential sleep stage that the user may be in during the current epoch. Thus, when applying the first set of coefficients in step 608, the coefficients that correspond to the detected event and each distinct potential sleep stage are the coefficients that are used. Applying the coefficients includes multiplying each sleep stage probability by the appropriate coefficient. Thus, if an apnea event was detected during the current epoch, step 608 includes: (i) multiplying the sleep stage probability for the wake stage by the coefficient corresponding to the apnea event and the wake stage, (ii) multiplying the sleep stage probability for the light sleep stage by the coefficient corresponding to the apnea event and the light sleep stage, (iii) multiplying the sleep stage probability for the deep sleep stage by the coefficient corresponding to the apnea event and the deep sleep stage, and (iv) multiplying the sleep stage probability for the REM sleep stage by the coefficient corresponding to the apnea event and the REM sleep stage. In general, if there are n different potential sleep stages for the current epoch (e.g., if step 606 includes determining n different sleep stage probabilities) and m different types of events that can occur during the current epoch, then the total number of coefficients in the first set of coefficients will be n×m.

In some implementations, each of the sleep stage probabilities is a decimal number between 0 and 1, and each coefficient of the first set of coefficients is a decimal number between 0 and 1. Once each of plurality of sleep stage probabilities have been adjusted by multiplying the sleep stage probabilities by the appropriate coefficient of the first set of coefficients, the sleep stage with the highest probability can be selected as the sleep stage for the current epoch.

If no events occurred during the current epoch, method 600 then determines if any events occurred during a prior epoch. If one or more events did occur during the prior epoch, method 600 proceeds from step 608 to step 610B, where each of the sleep stage probabilities is adjusted based at least in part on the one or more events that occurred during the prior epoch. In some implementations, the prior epoch is the epoch that immediately precedes the current epoch. In other implementations, the prior epoch can be the immediately preceding epoch, or within a plurality of epochs (e.g., 2, 3, 4, 5, or more epochs) preceding the current epoch. In some implementations, adjusting the plurality of sleep stage probabilities based on events occurring during the prior epoch includes applying the same first set of coefficients to the sleep stage probabilities as step 610A. For example, if an apnea event is detected in the prior epoch, the sleep stage probability for each sleep stage will be multiplied by the exact same coefficient as if the apnea event had been detected during the current epoch. In other implementations however, a different set of coefficients may be applied to the sleep stage probabilities if any events occurred during the prior epoch. In these implementations however, the different set of coefficients will still generally have a single distinct coefficient for each combination of sleep stage and event. However, the actual values of these coefficients will be different as compared to the first set of coefficients.

Finally, if no events occurred during either the current epoch or the prior epoch, method 600 then proceeds from step 608 to step 610C. At step 610C, each of the sleep stage probabilities is adjusted based at least in part on the determined sleep stage of the prior epoch, by applying a second set of coefficients to the sleep stage probabilities. Step 610C is similar to steps 610A and 610B, in that each sleep stage probability will be multiplied by a respective coefficient of the second set of coefficients. However, the coefficients of the second set of coefficients correspond to different combinations of (i) potential sleep stages of the current epoch and (ii) the determined sleep stage of the prior epoch, instead of different combinations of (i) potential sleep stages of the current epoch and (ii) events occurring during the current epoch or the prior epoch.

The second set of coefficients includes a distinct coefficient for each transition between the sleep stage of the prior epoch and the potential sleep stages of the current epoch. Thus, if there are n potential sleep stages that epochs can be categorized into, the second set of coefficients will include n² different coefficients. Each coefficient of the second set of coefficients corresponds to a distinct combination of a potential sleep stage for the current epoch, and a previously-determined sleep stage of the prior epoch. Similar to the first set of coefficients, each coefficient in the second set of coefficients is a decimal number between 0 and 1. Once each of plurality of sleep stage probabilities have been adjusted by multiplying the sleep stage probabilities by the appropriate coefficient of the second set of coefficients, the sleep stage with the highest probability can be selected as the sleep stage for the current epoch.

Thus, once the initial sleep stage probabilities are generated (for example by using a trained machine learning model), these sleep stage probabilities can be adjusted based on events experienced during the current epoch, events occurring during the prior epoch, or the determined sleep stage probability of the prior epoch. The sleep stage probabilities are adjusted by multiplying the sleep stage probabilities by an appropriate coefficient. Each coefficient will correspond to a distinct combination of (i) a potential sleep stage for the current epoch and an event occurring during the current epoch; (ii) a potential sleep stage for the current epoch and an event occurring during the prior epoch; or (iii) a potential sleep stage for the current epoch and a previously-determined sleep stage for the prior epoch.

In some implementations, method 600 can include determining whether the user interface was not worn by the user at any point during the current epoch. During the sleep session, the user interface may be detached from the user's head for a variety of reasons. For example, the user may remove the user interface to get up and use the restroom, or the user interface may inadvertently detach if the user moves around in their sleep. When this occurs, there will generally be gaps in the flow data and/or the respiration data. Method 600 can include identifying these gaps, and noting that the user interface was not worn by the user during at least a portion of the current epoch. When such an identification is made, the current epoch can be set as a Mask-On Mask-Off epoch (or “MOMO”), and the method can then proceed to analyze data for the next epoch within the sleep session.

In some implementations, method 600 can include generating a hypnogram in real-time as the sleep stages are determined for each epoch (e.g., as the sleep stage probabilities are generated and adjusted, and the highest adjusted sleep stage probability is selected as the sleep stage for each epoch). The hypnogram (which may be similar to hypnogram 400) can show the sleep stage for each epoch of the sleep session. The hypnogram may also indicate which epochs were designated as Mask-On Mask-Off epochs, if it was determined there was a gap in the data for those epochs.

In some implementations, method 600 can include filtering the epochs to smooth transitions between different epochs. In some cases, the hypnogram may include one or more abrupt transitions back and forth between sleep stages. For example, during the early portion of the sleep session, the hypnogram may oscillate back and forth between the wake stage and the light sleep stage. To smooth out this series of oscillations, the epochs can be filtered by adjusting the sleep stages of the epochs. A first portion of the sleep session that includes a group of distinct epochs can be selected, and the most common sleep stage for that group of epochs can be determined. The sleep stage of each epoch within that group of epochs can then be set to the most common sleep stage. The filtering process is continued by next selecting an entirely new portion of the sleep session containing a different group of distinct epochs, such that no single epoch is included in multiple separate groups.

The number of epochs within each group can, in some implementations, be based on the group of epochs occurs before sleep onset or after sleep onset. In some implementations, the number of epochs within each group before sleep onset is less than the number of epochs within each group after sleep onset. For example, the number of epochs in each group before sleep onset can be five epochs, and the number of epochs in each group after sleep onset can be seven or eight epochs. In some implementations, the sleep onset refers to only the initial onset of sleep during the sleep session. In other implementation, the sleep only refers to any onset of sleep. Thus, if the user wakes up during the sleep session (for example to use the restroom), the filtering process would begin to filter with the smaller number of epochs within each group, until it is determined that the user has fallen asleep again. This filter process generally corresponds to sub-block 550 of algorithm 500.

In some implementations, method 600 can include modifying the hypnogram in a variety of ways. These modifications generally correspond to the steps undertaken by block 516 of algorithm 500. In some implementations, method 600 includes identifying the initial epoch of the sleep session and the final epoch of the sleep session, and setting both of these epochs to the wake stage, if the wake stage was not the sleep stage with the highest adjusted sleep stage probability. In some implementations, method 600 includes identifying any epochs that were designated as Mask-On Mask-Off epochs. Then, both the sleep stage immediately prior to the Mask-On Mask-Off epoch and the sleep stage immediately after the Mask-On Mask-Off epoch can be set to the wake stage, if the wake stage was not the sleep stage with the highest adjusted sleep stage probability.

In some implementations, method 600 includes identifying consecutive epochs of the sleep session that represent invalid transitions between sleep stages, and either modifying the sleep stage of one or both of these epochs, or inserting an artificial epoch between these epochs. Invalid transitions between consecutive epochs can include (i) a wake stage to a deep sleep stage, (ii) a wake stage to an REM sleep stage, (iii) a deep sleep stage to an REM sleep stage, and (iv) an REM sleep stage to a deep sleep stage. In some implementations, an artificial epoch is inserted in between the two identified epochs, and the artificial epoch is set to a light sleep stage. In other implementations, the sleep stage of one or both of the two identified epochs can be modified, so that the two epochs no longer represent an invalid transition between sleep stages.

In some implementations, method 600 includes identifying a group of epochs that do not last for the minimum amount of time required for the designated sleep stage, and then modifying the sleep stage of those epochs. Generally, once the user reaches deep sleep or REM sleep, those sleep stages will last for at least some minimum amount of time. If a group of epochs set to a deep sleep stage or a REM sleep stage span a total amount of time that is less than the minimum amount of time, method 600 can include modifying the sleep stages of the epochs within the group of epochs. In some implementations, the sleep stage of each epoch within the group of epochs is set to the light sleep stage. In other implementations, artificial epochs can be added at any point within the group of epochs that are set to the same sleep stage, until the minimum amount of time is reached. In still other implementations, epochs prior to or after the group of epochs can be modified to be set to the same sleep stage as the group of epochs, until the minimum amount of time is reached.

Generally, method 600 can be implemented using a system having a control system with one or more processors, and a memory storing machine readable instructions. The controls system can be coupled to the memory, and method 600 can be implemented when the machine readable instructions are executed by at least one of the processors of the control system. Method 600 can also be implemented using a computer program product (such as a non-transitory computer readable medium) comprising instructions that when executed by a computer, cause the computer to carry out the steps of method 600.

One or more elements or aspects or steps, or any portion(s) thereof, from one or more of any of the claims below can be combined with one or more elements or aspects or steps, or any portion(s) thereof, from one or more of any of the other claims below or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

1. A system for determining a sleep stage of an individual, the system comprising:
an electronic interface configured to receive data associated with a sleep session of the individual;
a memory storing machine-readable instructions; and
a control system including one or more processors configured to execute the machine-readable instructions to:
receive the data associated with the sleep session of the individual, the sleep session being divided into a plurality of epochs;
analyze the received data to identify one or more features associated with a current epoch of the sleep session, the one or more features including (i) at least one feature associated with a flow of pressurized air from a respiratory therapy system used by the individual during the sleep session, (ii) at least one feature associated with a respiration rate of the individual, (iii) at least one feature associated with a temporal location of the current epoch within the sleep session, or (iv) any combination of (i)-(iii); and
determine, based on at least the one or more features, a plurality of sleep stage probabilities, each sleep stage probability corresponding to a respective one of a plurality of potential sleep stages during the current epoch of the sleep session.

2. The system of claim 39, wherein adjusting the plurality of sleep stage probabilities includes, in response to a number of events experienced by the individual during the current epoch of the sleep session or the prior epoch of the sleep session being greater than zero, multiplying each of the plurality of sleep stage probabilities by a respective coefficient of a first plurality of coefficients, a value of each coefficient of the first plurality of coefficients being based on a type of event experienced during the current epoch of the sleep session or a type of event experienced during the prior epoch of the sleep session.

3-4. (canceled)

5. The system of claim 2, wherein adjusting the plurality of sleep stage probabilities further includes, in response to the number of events experienced during the prior epoch of the sleep session being equal to zero, multiplying each of the plurality of sleep stage probabilities by a respective coefficient of a second plurality of coefficients.

6. The system of claim 5, wherein a value of each coefficient of the second plurality of coefficients is based on the previously-determined sleep stage for the prior epoch of the sleep session.

7-8. (canceled)

9. The system of claim 1, wherein the prior epoch occurs immediately before the current epoch, wherein the subsequent epoch of the sleep session occurs immediately after the current epoch of the sleep session, or both.

10. The system of claim 1, wherein each identified feature has a value, and wherein determining the plurality of sleep stage probabilities includes:
discarding features having outlier values;
standardizing the value of each remaining feature to an identical scale; and
inputting the standardized value of each remaining feature into a trained algorithm, the trained algorithm being configured to output the plurality of sleep stage probabilities.

11. The system of claim 1, wherein the one or more processors are further configured to execute the machine-readable instructions to:
select the one of the plurality of potential sleep stages having the highest sleep stage probability as the sleep stage for the current epoch of the sleep session; and
determine the sleep stage of each remaining epoch of the plurality of epochs based at least in part on identified features associated with each remaining epoch of the plurality of epochs.

12. The system of claim 11, wherein the one or more processors are further configured to execute the machine-readable instructions to:
divide the plurality of epochs of the sleep session into a plurality of groups of epochs, each group of epochs including two or more distinct epochs;
for each respective group of epochs, determine a most common sleep stage for the two or epochs in the respective group of epochs; and
for each respective group of epochs, set the sleep stage of the two or more epochs in the respective group of epochs to the most common sleep stage for the respective group of epochs.

13. The system of claim 12, wherein the one or more processors are further configured to execute the machine-readable instructions to:
identify a first portion of the sleep session that includes epochs occurring before the individual falls asleep during the sleep session; and
identify a second portion of the sleep session that includes epochs occurring after the individual initially falls asleep during the sleep session,
wherein any group of the plurality of groups of epochs containing epochs occurring before the individual falls asleep during the sleep session includes a first number of two or more distinct epochs, and
wherein any group of the plurality of groups of epochs containing epochs occurring after the individual initially falls asleep during the sleep session includes a second number of two or more distinct epochs that is different than the first number of two or more distinct epochs.

14-16. (canceled)

17. The system of claim 11, wherein the one or more processors are further configured to execute the machine-readable instructions to:
identify an initial epoch of the plurality of epochs;
identify a final epoch of the plurality of epochs; and
set the sleep stage of the initial epoch and the final epoch to a wake stage.

18. The system of claim 11, wherein the respiratory therapy system is configured to provide the pressurized air through a user interface wearable by the individual, and wherein the one or more processors are further configured to execute the machine-readable instructions to:
identify an epoch of the plurality of epochs where the user interface was not worn by the individual during at least a portion of the epoch;
set the sleep stage of the epoch immediately prior to the identified epoch to a wake stage; and
set the sleep stage of the epoch immediately after the identified epoch to a wake stage.

19. The system of claim 11, wherein the one or more processors are further configured to execute the machine-readable instructions to:
identify a first epoch of the plurality of epochs and a second epoch of the plurality of epochs, the second epoch occurring immediately after the first epoch, the first epoch and the second epoch representing an invalid transition between two distinct sleep stages;
insert an artificial epoch between the first epoch and the second epoch; and
set the sleep stage of the artificial epoch as a light sleep stage.

20. The system of claim 19, wherein:
the sleep stage of the first epoch is a wake stage, and the sleep stage of the second epoch is a deep sleep stage;
the sleep stage of the first epoch is a wake stage, and the sleep stage of the second epoch is a rapid eye movement (REM) sleep stage;
the sleep stage of the first epoch is a deep sleep stage, and the sleep stage of the second epoch is a rapid eye movement (REM) sleep stage; or
the sleep stage of the first epoch is a rapid eye movement (REM) sleep stage, and the sleep stage of the second epoch is a deep sleep stage.

21-23. (canceled)

24. The system of claim 11, wherein the one or more processors are further configured to execute the machine-readable instructions to:
identify a group of epochs set to a deep sleep stage or a rapid eye movement (REM) sleep stage;
determine a total amount of time spanned by the group of epochs; and
in response to the total amount of time spanned by the group of epochs being less than a predetermined minimum amount of time, set a sleep stage of each epoch of the group of epochs to a light sleep stage.

25-28. (canceled)

29. The system of claim 1, wherein the at least one feature associated with the flow of pressurized air includes (i) a maximum flow value across the current epoch and/or one or more prior epochs, (ii) a flow skew of the current epoch, (iii) a median flow skew across the current epoch and/or one or more prior epochs, (iv) a standard deviation of flow values for the current epoch, (v) a standard deviation of the standard deviation of flow values for the current epoch and/or one or more prior epochs, (vi) a flow volume stability across the current epoch and/or one or more prior epochs, (vii) a time ratio of inspiration to expiration for the current epoch, (viii) a ratio of inspiration volume to expiration volume for the current epoch, (ix) a maximum pressure during the current epoch and/or one or more prior epochs, (x) an average pressure during the current epoch and/or one or more prior epochs or (xi) any combination of (i)-(x).

30. The system of claim 1, wherein the at least one feature associated with the respiration rate of the individual includes (i) an average respiration rate across the current epoch and all prior epochs, (ii) a standard deviation of respiration rate values across the current epoch and one or more prior epochs, or (iii) both (i) and (ii).

31. The system of claim 1, wherein the at least one feature associated with the time of the sleep session includes (i) a number of the current epoch within the sleep session, (ii) the n^{th} root of the number of the current epoch within the sleep session, or (iii) both (i) and (ii).

32. The system of claim 31, wherein n=20.

33. (canceled)

34. The system of claim 1, wherein:
the one or more features includes (i) at least one feature associated with the flow of pressurized air from the respiratory therapy system used by the individual during the sleep session and (ii) at least one feature associated with the respiration rate of the individual;
the one or more features includes (i) at least one feature associated with the flow of pressurized air from the respiratory therapy system used by the individual during the sleep session and (ii) at least one feature associated with the temporal location of the current epoch within the sleep session;
the one or more features includes (i) at least one feature associated with the respiration rate of the individual and (ii) at least one feature associated with the temporal location of the current epoch within the sleep session; or
the one or more features includes (i) at least one feature associated with the flow of pressurized air from the respiratory therapy system used by the individual during the sleep session, (ii) at least one feature associated with the respiration rate of the individual, and (iii) at least one feature associated with the temporal location of the current epoch within the sleep session.

35-37. (canceled)

38. The system of claim 1,
wherein the system further comprises the respiratory therapy system configured to supply pressurized air to an individual.

39. The system of claim 1, wherein the one or more processors are further configured to execute the machine-readable instructions to:
analyze the data to identify events experienced by the individual during the current epoch of the sleep session; and
adjust each of the plurality of sleep stage probabilities based at least in part on (i) events experienced by the individual during the current epoch of the sleep session, (ii) events experienced by the individual during a prior epoch of the sleep session, (iii) events experienced by the individual during a subsequent epoch of the sleep session, (iv) a sleep stage determined for the prior epoch of the sleep session, (v) a sleep stage determined for the subsequent epoch of the sleep session, or (vi) any combination of (i)-(v).

40. (canceled)

41. A method of determining a sleep stage of an individual, the method comprising:
receiving data associated with a sleep session of the individual, the sleep session being divided into a plurality of epochs;
analyzing the received data to identify one or more features associated with a current epoch of the sleep session, the one or more features including (i) at least one feature associated with a flow of pressurized air from a respiratory therapy system used by the individual during the sleep session, (ii) at least one feature associated with a respiration rate of the individual, (iii) at least one feature associated with a temporal location of the current epoch within the sleep session, or (iv) any combination of (i)-(iii); and
determining, based on at least the one or more features, a plurality of sleep stage probabilities, each sleep stage probability corresponding to a respective one of a plurality of potential sleep stages during the current epoch of the sleep session.

42-80. (canceled)

81. A system for determining a sleep stage of an individual, the system comprising:
a control system including one or more processors; and
a memory having stored thereon machine-readable instructions;
wherein the control system is coupled to the memory, and wherein when the machine-readable instructions in the memory are executed by at least one of the one or more processors of the control system, a method is implemented that includes:
receiving data associated with a sleep session of the individual, the sleep session being divided into a plurality of epochs;
analyzing the received data to identify one or more features associated with a current epoch of the sleep session, the one or more features including (i) at least one feature associated with a flow of pressurized air from a respiratory therapy system used by the individual during the sleep session, (ii) at least one feature associated with a respiration rate of the individual, (iii) at least one feature associated with a temporal location of the current epoch within the sleep session, or (iv) any combination of (i)-(iii); and
determining, based on at least the one or more features, a plurality of sleep stage probabilities, each sleep stage probability corresponding to a respective one of a plurality of potential sleep stages during the current epoch of the sleep session.

82. A system for determining a sleep stage of an individual, the system including a control system having one or more processors configured to implement the method of claim 41.

83. A computer program product comprising instructions which, when executed by a computer, cause the computer to carry out the method of claim 41.

84. (canceled)
